(19) United States
(12) Patent Application Publication
(10) Pub. No.: US 2004/0109854 A1
(43) Pub. Date: Jun. 10, 2004

(54) Novel traf6 inhibiting protein

(76) Inventors: Zang-Hee Lee, Bongseon-dong (KR); Hong-Hee Kim, Bongseon-dong (KR)

Correspondence Address: Squire Sanders & Dempsey, 14th Floor, 801 Figueroa Street, Los Angeles, CA, 90017-5554, US

(21) Appl. No.: 10/203,052

(22) PCT Filed: Feb. 21, 2002
(86) PCT No.: PCT/KR02/00276

(30) Foreign Application Priority Data
Feb. 21, 2001 (KR) 2001-8595

(51) Int. Cl. C12Q 1/68; C07H 21/04; A61K 39/395; C12N 9/99

(52) U.S. Cl. 424/130.1; 435/6; 435/69.1; 435/184; 435/320.1; 435/325; 530/388.26; 536/23.2

(57) ABSTRACT

Disclosed are novel isolated tumor necrosis factor receptor associated factor (TRAF) 6 inhibiting proteins or functional derivatives thereof The proteins contain a KRAB domain at their N-termini and 14 C₂H₂ type zinc finger motifs at their C-termini, bind to TFAF6 but do not bind to TRAF2 and TRAF3, and inhibit activities of NF-κB or AP-1 in a signal transduction pathway of tumor necrosis factor (TNF) by interacting with TRAF6.

### TECHNICAL FIELD

The present invention relates to the isolation, recombinant production and characterization of novel polypeptides which inhibit tumor necrosis factor receptor associated factor 6 (hereinafter referred to as TRAF6) or block signal transduction mediated by TRAF6. In particular, this invention relates to novel proteins comprising a KRAB domain at their N-termini and fourteen C₂H₂ type zinc finger motifs at their C-termini, which bind to TFAF6 but do not bind to TRAF2 and TRAF3 and inhibit activity of TRAF6, NF-κB or AP-1, or functional derivatives thereof

### BACKGROUND ART

Tumor necrosis factor(TNF) is a cytokine produced mainly in activated macrophages. The cellular physiological effects of TNF are initiated by TNF binding to its two receptors: TNFR-1 of approximately 55 kDa and TNFR-2 of approximately 75 kDa. cDNA sequences of TNFR-1 and TNFR-2 have been isolated from human and mouse. These TNFRs are composed of an extracellular domain, a transmembrane domain and an intracellular domain, like other cell surface receptors. These TNFRs also belong to the TNFR superfamily along with CD40, etc. and mediate activation of the transcription factor NF-κB. The TNFR superfamily is expressed in most cell types and is involved in cellular proliferation and differentiation, inflammation response, growth of bone and apoptosis (Smith et al., Cell, 76, 959-962, 1994). NF-κB is a member of the Rel family of the transcription activators that control the activity of various important cellular and viral genes.

Members of the TNFR superfamily typically interact with a group of adaptor proteins known as TRAF in an intracellular region. TRAF proteins have a Ring and a zinc finger motif at their N-termini and a TRAF domain, which is a highly conserved sequence of TRAF proteins, at their C-termini (Arch et al., Genes Dev., 12, 2821-2830, 1998). So far, six types of TRAF proteins, including TRAF1 to TRAF6, have been identified. TRAF2, TRAF5 and TRAF6 have been disclosed to activate the NF-κB pathway and JNK (c-Jun N-terminal kinase) pathway by their over-expression, whereas the specific roles of TRAF1, TRAF3 and TRAF4 in signal transduction are not yet known (Kim et al., FEBS Lett., 443, 297-302, 1999; and Rothe et al., Science, 269, 1424, 1995).

TRAF6 is involved in gene activation mediated by TNFR and interleukin-1 (IL-1) receptors. Recently, Lomaga et al. have demonstrated that TRAF6 plays a critical role in NF-κB activation, dependant on CD40L, IL-1 and lipopolysacharide, in experiments using TRAF6 deficient mice (Lomaga et al., Genes Dev., 15, 1010-1024, 1999; and Naito et al., Genes Cells, 4, 353-362, 1999). TRAF6 interacts with a cytoplasmic domain of receptor activators of NF-κB (RANK) and mediates NF-κB activation induced by the RANK through a signal transduction pathway of TRAF/NF-κB-inducing kinase (NIK)/IκB kinase (IKK) (Darnay et al., J. Biol. Chem., 274, 7724-7731, 1999). IL-1 and TNF-α stimulate AP-1 and NF-κB transcription factors, respectively, through activation of JNK and p38 MAP kinases, and IKK. TNF-α and IL-1 transmit signals to cells through oligomerization of TRAF2 and TRAF6 (Veronique et al, Genes Dev., 13, 1297-1308, 1999), and TRAF6 interacts with IL-1 receptor-associated kinase 1 and 2 (IRAK 1 and IRAK 2). When 293 cells were treated with L-1, TRAF6 was found to bind to IRAK serine/threonine kinase, which was rapidly induced by the IL-1 receptor upon stimulation of IL-1. This suggests that TRAF proteins can act as a signal transduction factor for a certain group of receptors and TRAF6 is involved in IL-1 signal transmission (Cao et al, Nature, 383, 443446, 1996; and Muzio et al., Science, 278, 1612-1615, 1997).

There are more than 300 genes possessing a zinc finger domain in the human genome (Bellefroid et al., DNA, 8, 377-387, 1989). In general, a zinc finger region in a protein is composed of repeating zinc finger motifs in series. In some cases, a group of zinc fingers may be separated by a non-finger region from another group of zinc fingers and (Ruizi et al., EMBO, 6, 3065-3070, 1987; Fan and Maniatis, Genes Dev., 4, 29-42, 1990). Genes of zinc finger groups are divided into two groups according to the amino acid chelating zinc: one is a Kruppel-like protein or a TFIIIA-like protein which is characterized by having two cysteine residues and two histidine residues (C2H2) to coordinate a zinc ion, and the other is a hormone receptor having two pairs of cysteine residues (C2C2) (Gibson et al, Protein Eng., 2, 209-218, 1988; Berg J. M., Proc. Natl. Acad. Sci. USA, 85, 99-102, 1988; and Evans RM., Science, 240, 889-895, 1988). Most of the identified zinc finger proteins specifically bind to DNA and are transcription regulators (Green and Chambon, Nature, 325, 75-78, 1987; and Blumberg et al., Nature, 328, 443-445, 1987).

### DISCLOSURE OF THE INVENTION

The present invention is based on the identification, recombinant production and characterization of a certain group of novel TRAF6inhibiting proteins, which are herein referred to as “TRAF6-inhibiting proteins”. More specifically, the present invention is based on the isolation of cDNAs encoding various forms of human TRAF6-inhibiting proteins using a yeast two-hybrid system, with no significant sequence similarity to any known protein, and on the expression and characterization of the encoded TRAF6-inhibiting proteins.

In one aspect, the present invention provides novel TRAF6-inhibiting proteins comprising a KRAB domain at their N-termini and fourteen zinc finger motifs at their C-termini, which bind to TFAF6 but do not bind to TRAF2 and TRAF3 and inhibit the physiological activities mediated by TRAF6, NF-κB or AP-1. In particular, the present invention provides novel TRAF6-inhibiting proteins comprising the amino acid sequence shown in FIG. 6A. Also, the present invention provides functional derivatives of the novel TRAF6-inhibiting proteins comprising the amino acid sequence shown in FIG. 6A.

In another aspect, the present invention provides nucleic acid sequences encoding TRAF6-inhibiting proteins comprising a KRAB domain at their N-termini and 14 zinc finger motifs at their C-termini, which bind to TFAF6 but do not bind to TRAF2 and TRAF3, and inhibit the physiological activities mediated by TRAF6, NF-κB or AP-1. In particular, the present invention provides novel nucleic acid sequences comprising the nucleic acid sequence shown in FIG. 5. The nucleic acid sequences according to the present invention comprise gDNA, cDNA and RNA, and nucleic acid sequences encoding functional derivatives of TRAF6-inhibiting proteins.

In a further aspect, the present invention provides plasmids comprising the aforementioned nucleic acid sequences, for example, cloning vectors or recombinant plasmids expressing TRAF6-inhibiting proteins.

In yet another aspect, the present invention provides cells and cell lines comprising the vectors expressing TRAF6-inhibiting proteins.

In yet another aspect, the present invention provides a method for producing TRAF6-inhibiting proteins, and more preferably a method for producing TRAF6-inhibiting proteins by a genetic engineering technique.

In yet another aspect, the present invention provides a method for purifying a molecule capable of selectively binding to TRAF6-inhibiting proteins, for example, TRAF6 or a fragment thereof, using TRAF6-inhibiting proteins, particularly mass-produced TRAF6-inhibiting proteins.

In yet another aspect, the present invention provides a method for screening a substance capable of modulating activity of TRAF6-inhibiting proteins, particularly a method for screening a substance promoting or inhibiting the binding of TRAF6 to TRAF6-inhibiting proteins.

In yet another aspect, the present invention provides a pharmaceutical composition useful in treating or preventing diseases associated with hyperactivity of TRAF6, NF-κB or AP-1, comprising a therapeutically or prophylactically effective amount of an active ingredient selected from the group consisting of expression vectors of TRAF6-inhibiting proteins, isolated mRNAs of TRAF6-inhibiting proteins, TRAF6-inhibiting proteins, and mixtures of any two or more thereof, alone or in combination with any pharmaceutically acceptable carrier.

In yet another aspect, the present invention provides a method for treating or preventing diseases associated with hyperactivity of TRAF6, NF-κB or AP-1, comprising administering a composition comprising a therapeutically or prophylactically effective amount of an active ingredient selected from the group consisting of expression vectors of TRAF6-inhibiting proteins, isolated mRNAs of TRAF6-inhibiting proteins, TRAF6-inhibiting proteins, and mixtures of any two or more thereof, alone or in combination with any pharmaceutically acceptable carrier, to an animal.

In yet another aspect, the present invention provides a pharmaceutical composition useful in treating or preventing diseases associated with hypoactivity of TRAF6, NF-κB or AP-1, comprising a therapeutically or prophylactically effective amount of an active ingredient selected from the group consisting of antisense RNAs of TRAF6-inhibiting proteins, antibodies directed against TRAF6-inhibiting proteins, and mixtures of any two or more thereof, alone or in combination with any pharmaceutically acceptable carrier.

In yet another aspect, the present invention provides a method for treating or preventing diseases associated with hypoactivity of TRAF6, NF-κB or AP-1, comprising administering a composition comprising a therapeutically or prophylactically effective amount of an active ingredient selected from the group consisting of antisense RNAs of TRAF6-inhibiting proteins, antibodies directed against TRAF6-inhibiting proteins, and mixtures of any two or more thereof, alone or in combination with any pharmaceutically acceptable carrier, to an animal.

In yet another aspect, the present invention provides antibodies selectively recognizing TRAF6-inhibiting proteins.

In yet another aspect, the present invention provides pharmaceutical compositions for treating or preventing metabolic bone diseases comprising an active ingredient selected from a group consisting of expression plasmids of TRAF6-inhibiting proteins, isolated mRNAs of TRAF6-inhibiting proteins, TRAF6-inhibiting proteins, and mixtures of any two or more thereof.

In yet another aspect, the present invention provides a method for treating or preventing metabolic bone diseases, comprising administering a composition comprising an active ingredient selected from a group consisting of expression plasmids of TRAF6-inhibiting proteins, isolated mRNAs of TRAF6-inhibiting proteins, TRAF6-inhibiting proteins, and mixtures of any two or more thereof to an animal.

### BRIEF DESCRIPTION OF THE DRAWINGS

The above objects, and other features and advantages of the present invention will become more apparent after a reading of the following detailed description when taken in conjunction with the drawings, in which:

FIG. 1A is a map of the full length bait vector pGBT9-TRAF6 constructed according to the present invention;

FIG. 1B is a map of the bait vector pGBT9-TRAF6 dm constructed according to the present invention;

FIG. 2A is a map of the recombinant plasmid pRK-Flag-TBZF constructed by cloning the full length cDNA of TRAF6-inhibiting proteins to the vector pRK-Flag according to the present invention;

FIG. 2B is a map of the recombinant plasmid pSR α NtHA-TRAF6 constructed by cloning the fill length cDNA of TRAF6-inhibiting proteins to the vector pSR α NtHA according to the present invention;

FIG. 3A is a map of the full length recombinant plasmid pM-TRAF6 constructed by subcloning the full length TRAF6 cDNA to the vector pM containing the GAL4 DNA binding domain (DB) according to the present invention;

FIG. 3B is a map of the fill length recombinant plasmid pVP16-TBZF constructed by subcloning the full length TRAF6 cDNA to the vector pVP16 containing the VP 16 transcription activation domain (AD) according to the present invention;

FIG. 4 is a bar graph showing levels of interaction between TRAF6 deletion mutant (amino acids 1 to 274) and TBZF in yeast;

FIG. 5 shows the nucleotide sequence of the full length cDNA of TRAF6-inhibiting proteins according to the present invention;

FIG. 6A shows the full length amino acid sequence of TRAF6-inhibiting proteins according to the present invention deduced from the nucleotide sequence shown in FIG. 5 and the important characteristic parts thereof Italic letters represent the KRAB domain, underlined parts represent the cloned parts obtained from the yeast two-hybrid screening method, and darkened parts represent the zinc finger domain;

FIG. 6B and FIG. 6C show the comparison of the amino acid sequence of the TRAF6-inhibiting proteins shown in FIG. 6A and the amino acid sequence of the human zinc finger protein 85 (znf 85);

FIG. 7 is a photograph showing results of Western blot analyses in which the blast cell fraction is reacted with anti-TRAF6 antibodies, anti-Flag antibodies and anti-tubline antibodies. TBZF represents another nomenclature of the TRAF6-inhibiting proteins according to the present invention;

FIG. 8 is a photograph showing results of a Western blot analysis in which the interaction between TRAF6 and TRAF6-inhibiting proteins was examined in a mammal;

FIG. 9A to FIG. 9D are diagrams showing the effects of the TRAF6-inhibiting proteins according to the present invention on the activations of NF-κB (A, C, D) and AP-1 (B) by TRAF6 (A and B), RANK (C) and IL-1R (D). TBZF represents another nomenclature of the TRAF6-inhibiting proteins according to the present invention;

FIG. 10A and FIG. 10B are diagrams showing the effects of the TRAF6-inhibiting proteins according to the present invention on NF-κB activation by TNF and IL-1β. TBZF represents another nomenclature of the TRAF6-inhibiting proteins according to the present invention;

FIG. 11 is a map of the recombinant plasmid pCR2.1TOPO-TBZF expressing the TRAF6-inhibiting proteins according to the present invention;

FIG. 12 is a map of the recombinant plasmid pGEX5X-3-TBZF expressing the TRAF6-inhibiting proteins according to the present invention-GST fusion proteins for use as an immunogen in preparation of antibodies;

FIG. 13 is a photograph obtained using a confocal microscope (400X) showing the intracellular distribution of the TRAF6 and TRAF6-inhibiting proteins expressed in the COS cell lines. Photograph (a), using a normal optical microscope, shows the general shape of a cell; photograph (b), using a confocal microscope, shows weak signals of the TRAF6-inhibiting proteins expressed in nuclei; photograph (c), using a confocal microscope, shows the distribution of the GFP tagged TRAF6 in the cytoplasmic region near the nuclei; and photograph (d), using a confocal microscope, shows that the TRAF6-inhibiting proteins and the TRAF are co-located in separate intracellular regions; and

FIG. 14A and FIG. 14B are photographs obtained using an optical microscope (100×) of the TRAP-stained Raw264.7 cell demonstrating that the over-expression of TRAF6 inhibits differentiation of Raw264.7 cells into osteoclasts and a diagram showing results of quantification of osteoclast, respectively.

### BEST MODE FOR CARRYING OUT THE INVENTION

The present invention will now described in detail.

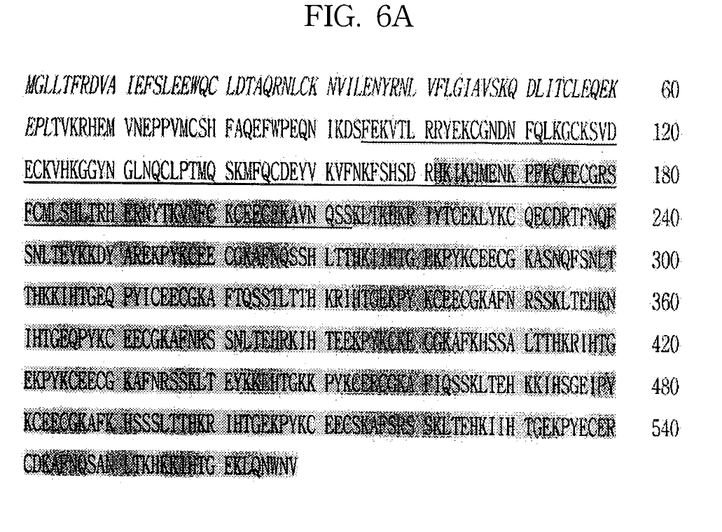

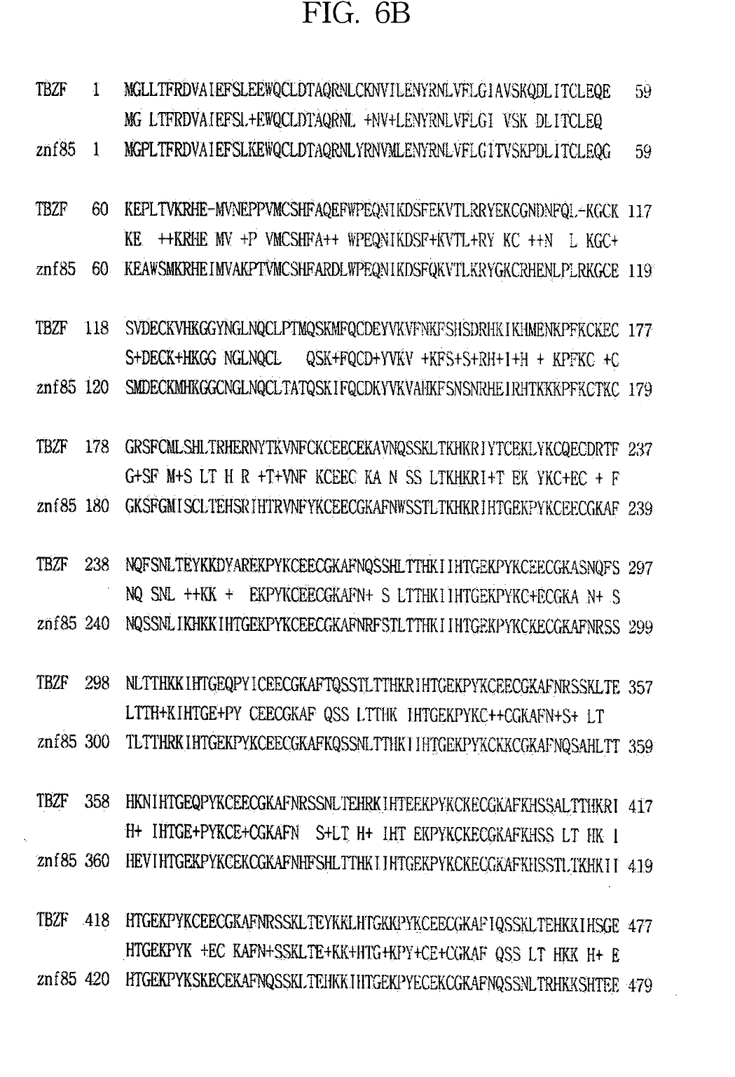

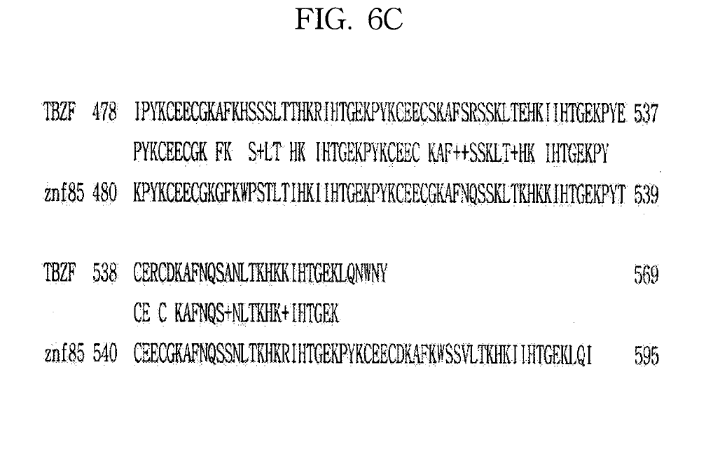

The phrases “TRAF6-inhibiting protein” used herein refers to an isolated native sequence polypeptide which interacts with TRAF6, thereby inhibiting physiological activities mediated by TRAF6, NF-κB or AP-1. According to the present invention, the TRAF6-inhibiting proteins are characterized in that (i) they bind to TFAF6 but do not bind to TRAF2 and TRAF3; (ii) they inhibit the physiological activities mediated by TRAF6, NF-κB or AP-1; and (iii) they contain a KRAB domain at their N-termini and 14 zinc finger motifs at their C-termini. According to the present invention, the TRAF6-inhibiting proteins comprise particularly a polypeptide having the sequence of 569 amino acids shown in FIG. 6A and splicings and allelic variants thereof.

The phrases “native polypeptide” used herein designates a polypeptide as occurring in nature in any cell type of any human or non-human animal species, with or without the initiating methionine, whether purified from native source, synthesized, produced by recombinant DNA technology or by any combination of these and/or other methods. From this point of view, native polypeptide from other mammalian species, such as porcine, canine, equine, etc. are also included in the present invention.

The TRAF6-inhibiting proteins according to the present invention comprise functional derivatives thereof A “functional derivative” of a native polypeptide is a compound having a biological activity in common with the native polypeptide. For the purpose of the present invention, for “functional derivatives” of native TRAF6-inhibiting proteins to have a biological activity in common with the native TRAF6-inhibiting proteins means that the functional derivative binds to TFAF6 but does not bind to TRAF2 and TRAF3 and inhibits the physiological activities mediated by TRAF6, NF-κB or AP-1. Preferably, functional derivatives of the native TRAF6-inhibiting protein according to the present invention are functional derivatives of the polypeptide having the amino acid sequence shown in FIG. 6A. The functional derivatives preferably have at least about 60%, more preferably at least about 70%, even more preferably at least about 80%, most preferably at least about 90% overall amino acid sequence identity with a native sequence TRAF6-inhibiting protein, preferably a human TRAF6-inhibiting protein. Even more preferably, the functional derivatives show at least about 70%, more preferably at least about 80% and most preferably at least about 90% amino acid sequence identity with the TRAF6-binding domain of a native sequence TRAF6-inhibiting protein.

As examples of the functional derivatives of the TRAF6-inhibiting proteins according to the present invention, compounds in which zinc finger motifs in the proteins are substituted with other known zinc finger motifs diversely existing in cells are included.

Also, the functional derivatives of the TRAF6-inhibiting proteins according to the present invention comprise amino acid sequence variants in which a part of the native protein is substituted, deleted or inserted, as long as the inherent biological activities are not lost. The substitutions of amino acids are preferably conservative substitutions. Examples of conservative substitutions of amino acids occurring in nature are as follows: aliphatic amino acids (Gly, Ala, Pro); hydrophobic or aromatic amino acids (Ile, Leu, Val, Phe, Tyr, Trp); acid amino acids (Asp, Glu); basic amino aicds (His, Lys, Arg, Gln, Asn); and sulfur-containing amino aicds (Cys, Met). Deletions of amino acids are preferably at positions where the TRAF6-inhibiting proteins do not interact with TRAF6, or which are not directly involved in the activity of the zinc finger motif.

Polypeptides which are fragments of native TRAF6-inhibiting proteins from various mammalian species and have a biological activity substantially in common with the native TRAF6-inhibiting proteins constitute another preferred group of functional derivatives of the native TRAF6-inhibiting proteins. The term “fragment” is an amino acid sequence corresponding to a part of a protein, which shares original elements, structures and functional mechanisms within the scope of the present invention, and comprises any of them as occurring in nature, or cut using protease or by chemical means. Also, functional derivatives having been modified to change the stability, shelf life, solubility, etc. of the native TRAF6-inhibiting proteins and to change the relation native protein's functional relationship with a substance interacting with the TRAF6-inhibiting protein, for example TRAF6, constitute another preferred group of functional derivatives of the native TRAF6-inhibiting proteins.

Another preferred group of functional derivatives of the native TRAF6-inhibiting proteins includes polypeptides encoded by a DNA sequence hybridizing under stringent conditions to the complement of a DNA sequence encoding a native TRAF6-inhibiting protein.

The term “Identity” or “homology” used herein with respect to a native protein or its functional derivatives is the percentage of amino acid residues in the candidate sequence that are identical with the residues of a corresponding native protein, after aligning the sequences and introducing gaps, if necessary, to achieve maximum homology, and not considering any conservative substitutions as part of the sequence identity. Methods and computer programs for the alignment are well known in the art.

“Stringent conditions” can be provided in a variety of ways, such as by overnight incubation at 42° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 μg/m denatured, sheared salmon sperm DNA. Alternatively, the stringent conditions can be achieved by a hybridization buffer comprising 30% formamide in 5×SSPE (0.18 M NaCl, 0.01 M NaPO₄, pH 7.7, 0.0001 M EDTA) buffer at a temperature of 42° C., and subsequent washing at 42° C. with 0.2×SSPE. Preferably, the stringent conditions involve the use of a hybridization buffer comprising 50% formamide in 5×SSPE at a temperature of 42° C. and washing at the same temperature with 0.2×SSPE.

The term “isolated” used herein with respect to a protein or a polypeptide or a nucleic acid means that the protein or polypeptide or nucleic acid is not accompanied with at least some of the material with which it is associated in its native environment. An isolated protein or polypeptide constitutes at least about 2% by weight and preferably at least about 5% by weight of the total protein in a given sample. An isolated nucleic acid constitutes at least about 0.5% by weight and preferably at least about 5% by weight of the total nucleic acid present in a given sample.

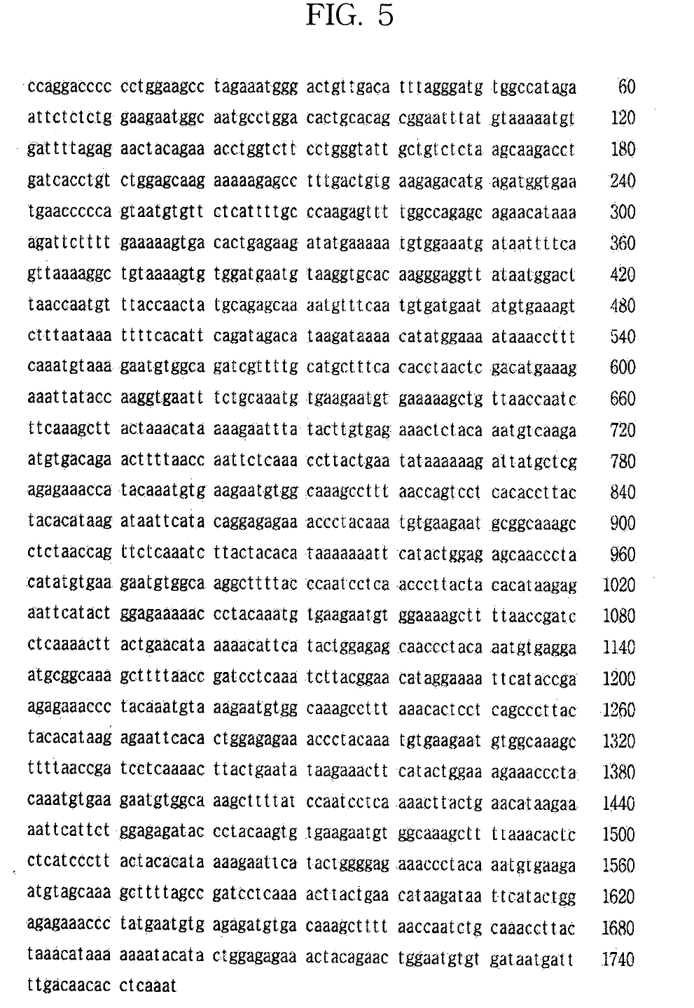

The present inventors screened a human cDNA library by yeast two-hybrid screening using TRAF dm (1-274 aa) which is a part of human TRAF6, as a bait As a result a clone was obtained which more strongly binds to TRAF6. Also, cDNA of the positive clone was collected and analyzed for its sequence to obtain the sequence shown in FIG. 5 (SEQ ID NO. 6). According to the sequence analysis by Genebank BLAST homology search system, the sequence is found to be a novel protein which was not yet known. The novel TRAF6-inhibiting proteins according to the present invention contain a KRAB domain and 14 zinc finger repeating units and shows the highest homology with human zinc finger protein 85 (FIG. 6B and FIG. 6C). Many members belonging to the human zinc finger protein superfamily comprise a transcription inhibiting factor domain, a KRAB domain and repeating zinc finger motifs, and are confirmed to inhibit transcription through specific binding to DNA. Recently, a group of researchers identified novel zinc finger proteins which bind to a target protein to inhibit its function (Karen and Rudi, FEBS Letters, 442, 147-150, 1999; Lei et al., Molecular Cell, 6, 757-768, 2000; and Song et al., Proc. Natl. Acad. Sci. USA, 93, 6721-6725, 1996). However, the TRAF6-inhibiting proteins of the present invention are substantially different from known proteins in their amino acid sequences. The presence of functional motifs (the KRAB and zinc finger domain) in the sequence coding the TRAF6-inhibiting proteins of the present invention apparently suggests that the TRAF6-inhibiting proteins of the present invention are transcription factors. The TRAF6-inhibiting proteins according to the present invention are of zinc finger C2H2 type.

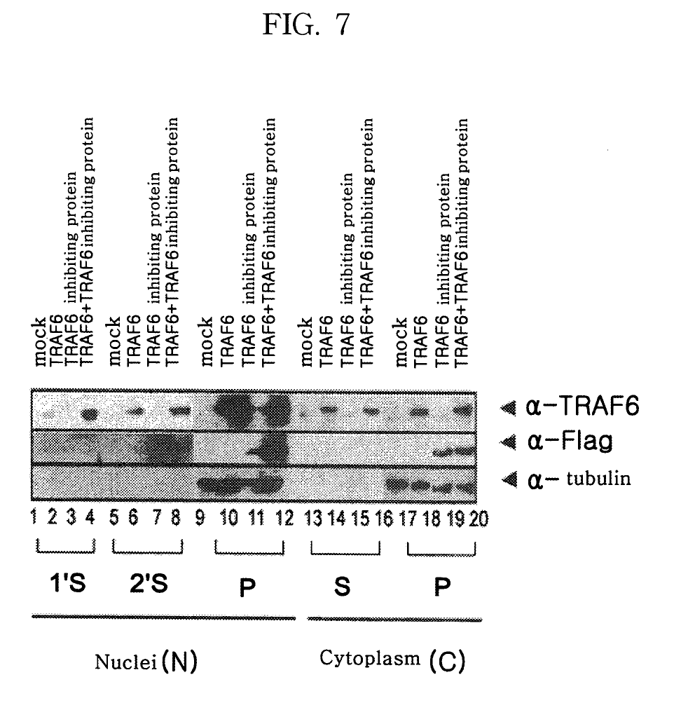

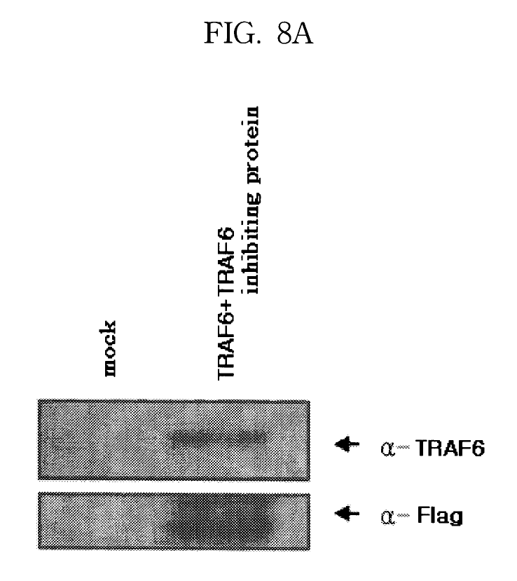

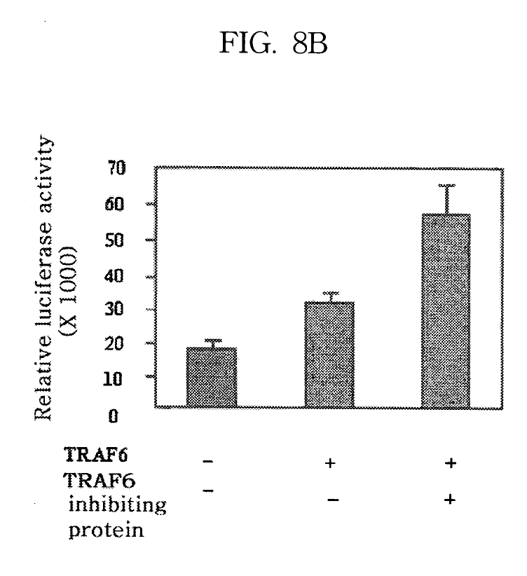

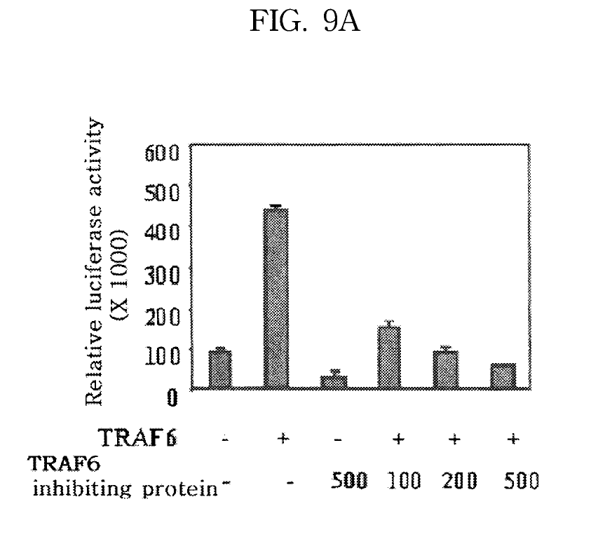

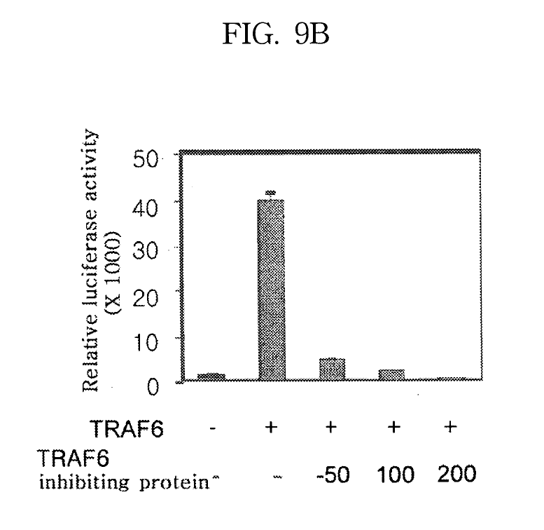

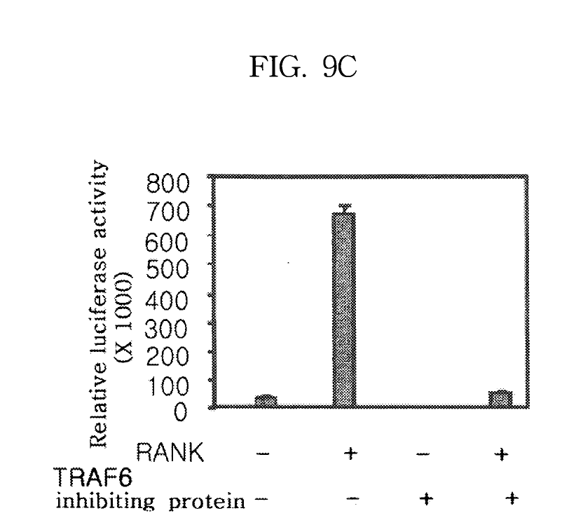

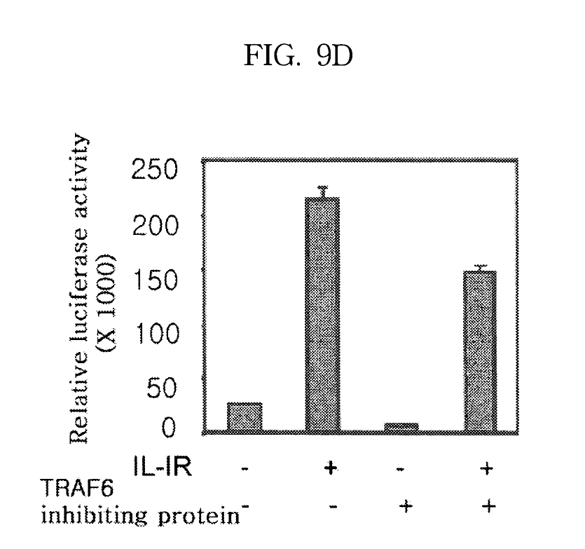

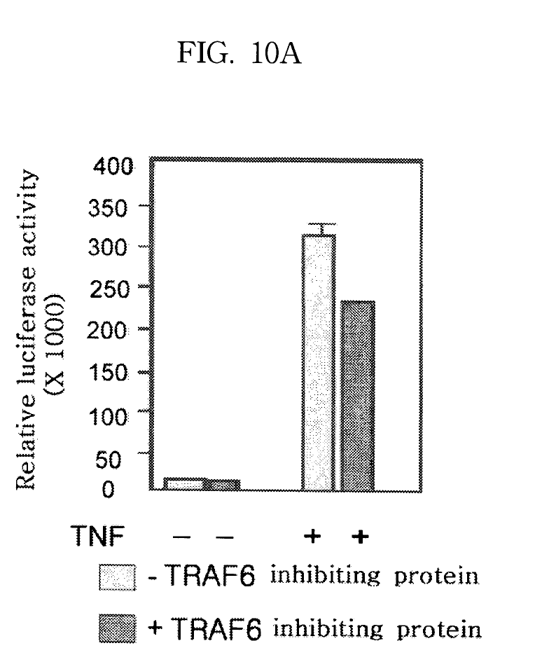

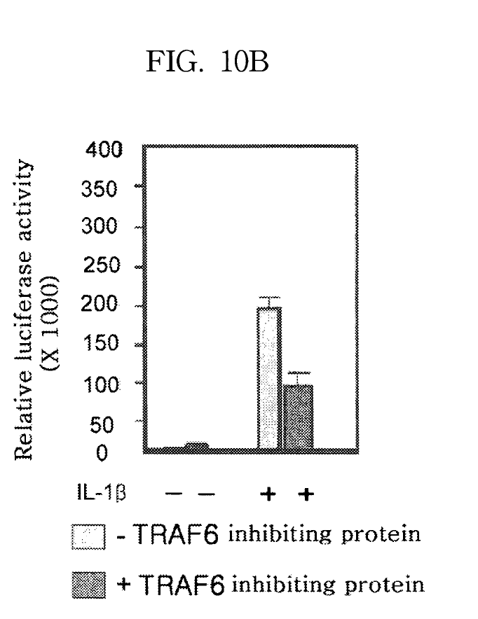

In the analysis of the TRAF6-inhibiting proteins of the present invention by in vitro yeast two-hybrid experiment, it was shown that the TRAF6-inhibiting proteins selectively bind to TRAF6 but do not bind to TRAF 2, TRAF 3, or other LRRY and pGBT 9 expression products (Table 3). Furthermore, upon examination of protein distribution in fractionated 293T cells, it was shown that the TRAF6-inhibiting proteins and TRAF6 are located in the same blast cell region (FIG. 7). In a coimmnunoprecipitation experiment of 293T cells transformed with gene of the TRAF6-inhibiting proteins, the same result is observed (FIG. 8A). From the presence of the zinc finger and the experimental results demonstrating that the over-expression of the TRAF6-inhibiting proteins can block the activation of NF-κB by TRAF6, RANK and IL-1β and the activation of AP-1 by TRAF6 (FIG. 9A to FIG. 10B), it is proven that the TRAF6-inhibiting proteins according to the present invention are critical factors modifying functions of TRAF6 in the TNF signaling pathway, particularly a factor inhibiting the activities of NF-κB and AP- 1.

The TRAF6-inhibiting proteins can be identified and purified from tissues expressing their MRNA, based on their ability to bind to TRAF6. Examples of the tissues include, but are not limited to, lung, liver, skeletal muscle, spleen, brain, kidney, etc. Native TRAF6-inhibiting proteins will be coprecipitated with immunoprecipitated TRAF6. In a preferred embodiment, radiolabeled TRAF6 is immunoprecipitated with protein A-agarose (Oncogene Science) or with protein A-Sepharose (Pharmacia). The immunoprecipitate is then analyzed by autoradiography or fluorography, depending on the radiolabel used. The TRAF6-inhibiting proteins will be coprecipitated with TRAF6 or its derivatives, and can be further purified by methods known in the art, such as purification on an affinity column. For large-scale purification, a scheme similar to that described by Smith and Johnson, Gene 67, 31-40 (1988) can be used. A cell lysate containing the TRAF6-inhibiting protein(s) to be purified is applied to a glutathione-S-transferase (GST)-TRAF6 fusion protein affinity column. Protein(s) bound to the column is/are eluted, precipitated and isolated by SDS-PAGE under reducing conditions, and visualized, e.g. by silver staining.

The TRAF6-inhibiting proteins, functional derivatives, fragments and any variants thereof can be prepared by any one of known organic chemistry methods for peptide synthesis. Organic chemistry methods for peptide synthesis comprise coupling a needed amino acid by condensation in a homogeneous phase or by the aid of a so-called solid phase. Common methods for condensation include, for example, the carbodiimide method, azide method, mixed anhydrides method and activated ester method, as disclosed in The Peptides Analysis, Synthesis, Biology Vol. 1-3 (Gross, E. and Meienhofer, J. ed.), 1979-1981 (Academic Press Inc.).

A particularly suitable solid phase includes p-alkoxybenzyl alcohol resin (4-hydroxy-methyl-phenoxy-methyl-copolystyrene-1% divinylbenzene resin) described in Wang, J. Am. Chem. Soc., 95, 132, 1974. Synthesized peptides can be isolated from the solid phase under mild conditions. After synthesis of a desired amino acid sequence, the separation of the produced peptide is performed using trifluoroacetic acid containing a scavenger such as triisopropyl silane, anisol, or ethanedithiol, thioanisol. A reactive radical capable of not participitating in the condensation is effectively protected by a group which can be readily removed by hydrolysis or reduction with an acid or base. More detailed description of the useful protective groups is given in The Peptides Analysis, Synthesis, Biology Vol. 1-3 (Gross, E. and Meienhofer, J. ed), 1979-1981 (Academic Press Inc.).

Particlarly preferred TRAF6-inhibiting proteins, functional derivatives thereof, fragments and any variants above described can be prepared using a gene recombinant technique. Genes of native TRAF6-inhibiting proteins are expressed in an appropriate host cell, which is then lysed to produce the lysate of the host cell. Alternatively, mRNAs of TRAF6-inhibiting proteins are translated in vitro. Then, the translation products or lysates are subjected to a protein separation method known in the art to recover the proteins. Typically, the translation products or lysates are centrifuged to remove particulate cell debris and then subjected to a purification method such as precipitation, dialysis, various column chromatographies and the like. Examples of the column chromatographies include ion-exchange chromatography, gel-permeation chromatography, HPLC, reverse phase HPLC, preparative SDS-PAGE, chromatography on an affinity column. The affinity column can be prepared using for example, anti-TRAF6-inhibiting protein antibodies or TRAF6. Also, the TRAF6-inhibiting proteins can be recovered from a culture medium in which the TRAF6-inhibiting proteins are expressed as secretory proteins. General techniques of recombinant DNA technology are, for example, disclosed in Sambrook et al., Molecular Cloning: A laboratory Manual, Second Edition (Cold Spring Harbor, N.Y.: Cold Spring Harbor Laboratory Press) 1989.

Amino acid sequence variants of native TRAF6-inhibiting proteins including functional derivatives thereof are prepared by methods known in the art by introducing appropriate nucleotide changes into DNA of native TRAF6-inhibiting proteins, or by in vitro synthesis of the desired polypeptide. There are two principle variables in the constructions of amino acid sequence variants: the location of the mutation site and the nature of the mutation. With the exception of naturally-occuring alleles, which do not require the manipulation of the DNA sequence encoding the TRAF6-inhibiting proteins, the amino acid sequence variants of TRAF6-inhibiting proteins are preferably constructed by mutating the DNA, either to arrive at an allele or an amino acid sequence variant that does not occur in nature. Methods for identifying target residues within native proteins and for making amino acid sequence variants are well known in the art, and are, for example, disclosed in U.S. Pat. No. 5,108,901 issued Apr. 28, 1992. The preferred techniques include alanine-scanning mutagenesis, PCR mutagenesis, cassette mutagenesis, and the like, details of which are also found in general textbooks, such as, for example, Sambrook et al., Molecular Cloning: A laboratory Manual, Second Edition (Cold Spring Harbor, N.Y.: Cold Spring Harbor Laboratory Press) 1989 and Current Protocols in Molecular Biology, Ausubel et al eds., Greene Publishing Associates and Wiley-lnterscience 1991.

The present invention provides nucleic acid sequences encoding TRAF6-inhibiting proteins, shown in FIG. 6A, which bind to TRAF6, a subtype of TRAF, but do not bind TRAF 2 and TRAF 3, are capable of inhibiting physiological activities mediated by TRAF6, NF-κB, or AP-1, and contain a KRAB domain at their N-termini and 14 zinc finger motifs at their C-termini. The nucleic sequence comprises GDNA, cDNA and RNA. Also, the nucleic sequence comprises a nucleic acid sequence encoding a native protein, a functional equivalent and functional derivative thereof and a sequence capable of hybridizing to these sequences under stringent conditions. The stringent conditions are the same as defined in Molecular Cloning, Cold Spring Harbor, N.Y., Cold Spring Harbor Laboratory Press, 1989.

Nucleic acid sequences encoding TRAF6-inhibiting proteins are of mammalian origin, preferably human origin. More preferably, the nucleotide sequences encoding TRAF6-inhibiting proteins are the nucleotide sequences encoding proteins of the amino acid sequence shown in FIG. 6A (SEQ ID NO: 7) by codon degeneracy, most preferably the nucleotide sequence shown in FIG. 5 (SEQ ID NO: 6). The TRAF family is a group of adaptors interacting with TNF commonly known, in the art, which contain a Ring and a zinc finger motif at their N-termini and a TRAF domain, which is a highly conserved sequence shared with other TRAF proteins, at their C-termini. Therefore, for the purpose of the present invention, the nucleic acid sequences encoding TRAF6-inhibiting proteins may, for example, be obtained from cDNA libraries prepared from a tissue believed to possess TRAF MRNA or MRNA of TRAF6-inhibiting proteins and to express it at a detectable level. For example, a cDNA library can be constructed by obtaining polyadenylated MRNA from a cell line known to express a TRAF6 protein, and using the mRNA as a template to synthesize double stranded cDNA. Also, DNA encoding TRAF6-inhibiting proteins can be obtained from genomic libraries such as a human genomic cosmid library.

Libraries, either cDNA or genomic, are screened with probes designed to identify the gene of interest or the protein encoded by it For cDNA expression libraries, suitable probes include monoclonal and polyclonal antibodies that recognize and specifically bind to the TRAF6-inhibiting proteins. For cDNA libraries, suitable probes include carefully selected oligonucleotide probes (usually of about 20-80 bases in length) that encode a portion of TRAF6-inhibiting polypeptides from the same or different species. Appropriate probes for screening genomic DNA libraries include, but are not limited to, oligonucleotides, cDNAs, or fragments thereof that encode the same or a similar gene, and/or homologous genomic DNAs or figments thereof. Screening the cDNA or genomic library with the selected probe may be conducted using standard procedures as described in Sambrook et al., Molecular Cloning: A Laboratory Manual, New York, Cold Spring Harbor Laboratory Press, (1989).

cDNAs encoding TRAF6-inhibiting proteins can also be identified and isolated by other known techniques of recombinant DNA technology, such as by direct expression cloning or by using the polymerase chain reaction (PCR) as described in Current Protocols in Molecular Biology, Ausubel et al eds., Greene Publishing Associates and Wiley-lnterscience 1991. This method requires the use of oligonucleotide probes that hybridize to DNAs encoding TRAF6-inhibiting proteins.

As illustrated in the present invention, the nucleic acid sequences encoding TRAF6-inhibiting proteins can be obtained by using the yeast two-hybrid system (Fields and Song, Nature (London) 340, 245-246 (1989); Chien et al., Proc. Natl. Acad. Sci. USA 88, 9578-9582 (1991); and Chevray and Nathans, Proc. Natl. Acad. Sci. USA 89, 5789-5793 (1992)). The yeast two-hybrid system employs two hybrid proteins. Many transcription activators, such as yeast GALA, consist of two physically discrete modular domains, one acting as the DNA-binding domain, while the other one finctioning as the transcription activation domain. A deletion mutant of TRAF6, preferably TRAF6 dm (1-274 aa), its deletion mutants thereof is fused to the DNA-binding domain of GAL4 (the bait plasmid), and proteins to be screened are fused to the activation domain of GAL4 (prey plasmid). Once the GAL4 activity is reconstituted via protein-protein interaction, a GAL1-lacZ reporter gene is expressed under control of a GAL4-activated promoter. Colonies containing interacting polypeptides are then detected by measuring β-galactosidase activity. The yeast two-hybrid system is applied to a kit (MATCHMAKER™) commercially available from Clontech. The present inventors screened human cDNA libraries by the yeast two-hybrid screening method using TRAF6 dm (1-274aa), a part of human TRAF6, as bait From the screening, a clone which binds specifically to TRAF6 was obtained. We sequenced the positive clone and obtained the nucleotide sequence shown in FIG. 5 (SEQ ID NO: 6).

Once the gene sequence of the TRAF6-inhibiting proteins is known, the gene can also be obtained by chemical synthesis, following one of the methods described in Engels and Uhlmann, Angew. Chem. Int. Ed. Engl. 28, 716 (1989). These methods include triester, phosphite, phosphoramidite and H-phosphonate methods, PCR and other autoprimer methods, and oligonucleotide syntheses on solid supports.

The present invention provides vectors containing the nucleic acid sequences encoding the TRAF6-inhibiting proteins, for example, cloning vectors or expression vectors expressing TRAF6-inhibiting proteins. A “vector” as defined for the purpose of the present invention refers to a DNA construct containing a DNA sequence which is operably linked to a regulatory sequence capable of effecting the expression of the DNA in a suitable host cell and other DNA sequences which are introduced to facilitate the gene manipulation or to optimize the expression of the DNA. Such regulatory sequences include a promoter to effect transcription, an optional operator sequence to control such transcription, a sequence encoding suitable MRNA ribosome binding sites, and sequences which control the termination of transcription and translation. The vector may be a plasmid, a virus, a phage particle, or simply a potential genomic insert. Vectors particularly described in the present invention include a cloning vector such as pCR2.1 TOPO-TBZF and an expression vector such as pVP16-TBZF. Here, TBZF refers to the gene encoding the TRAF6-inhibiting proteins according to the present invention.

However, in order to express a DNA sequence encoding the TRAF6-inhibiting protein any of various expression regulatory sequences can be used as a vector. Examples of useful expression regulatory sequences include for example, the early and late promoters of SV40 or adenovirus, the lac system, the trp system the tac or trc systems, the T3 and T7 promoters, the major operator and promoter regions of phage λ, the control region of fd coat protein the promoter for 3-phosphoglycerate kinase or other glycolytic enzymes, the promoters of acid phosphatase, e.g., Pho5, the promoters of the yeast α-mating factors, and other sequences known to control the expression of genes of prokaryotic or eukaryotic cells and their viruses or combinations thereof.

A nucleic acid is “operably linked” to another nucleic acid when they are arranged in a functional relationship. This means that an appropriate molecule (for example, a transcription activator) binds to a regulatory sequence(s), a gene or a regulatory sequence(s) linked in such a way that the expression of the gene is modulated. For example, when DNA for a pre-sequence or secretory leader is expressed as a preprotein participating in secretion of polypeptide, the DNA is operably linked to the polypeptide, when a promoter or enhancer affects trascription of a sequence, the promoter or enhancer is operably linked to the coding sequence, when a ribosomal binding site affects transcription of a sequence, the ribosomal binding site is operably linked to the coding sequence, or when a ribosomal binding site is arranged to facilitate translation, the ribosomal binding site is operably liked to the coding sequence. However, the enhancer does not need to contact The linkage of these sequences are effected by ligation (linkage) in a convenient restriction enzyme site. If such a site does not exist, a conventioanlly synthesized oligonucleotide adaptor or linker may be used.

The present invention provides a host cell to cell lines (herein after referred to as host cells) containing the above-described vector. The host cells transformed or transfected by the above-described expression vector forms another aspect of the present invention. The term “transformation” used herein refers to a phenomenon wherein DNA becomes replicable in a form other than a chromosome or integration into a chromosome. The term “transfection” used herein refers to a phenomenon that an expression vector is received by the host cells whether or not any coding sequence is expressed in practice. The host cells according to the present invention may be eukaryotic or prokaryotic cells. Host cells with a high efficiency of DNA introduction and DNA expression are commonly used. Examples of host cells which can be used in the present invention include known eukaryotic and prokayotic host cells such as E. coli, Pseudomonas, Bacillus, Streptomyces, fungi and yeast; insect cells such as those derived from Spodoptera frugiperda (SF9); animal cells such as CHO and those derived from a mouse; African green monkey cells such as COS1, COS7, BSC1, BSC40 and BMT10; and tissue cultured human cells. Preferably, host cells which can be used in the present invention are yeast or animal cells, more preferably 293T cells or 293/EBNA cells.

Based on their ability to bind to TRAF6, the TRAF6-inhibiting proteins of the present invention can be used to isolate and purify native TRAF6 proteins or functional derivatives or various mutants and fragments thereof, which, in turn, are useful for the purification of the TNF receptor super family members (for example, TNF, CD40) to which they specifically bind. Therefore, in another aspect, the present invention provides a method for isolating and/or purifying a molecule binding selectively to the TRAF6-inhibiting proteins, for example TRAF6 proteins, or derivatives, mutants or fragments thereof, using the TRAF6-inhibiting proteins, particularly mass-expressed TRAF6-inhibiting proteins. The method comprises culturing a mixture of TRAF6-inhibiting proteins and a candidate molecule, isolating a complex, and removing the TRAF6-inhibiting proteins from the isolated complex. The isolation of the complex can be performed by commonly used isolation methods. For example, an affinity column prepared by immobilizing TRAF6-inhibiting proteins, an affinity column using anti-idio type antibodies, a yeast two-hybrid system.

The TRAF6-inhibiting proteins of the present invention can be used in assays for identifying leader compounds of therapeutically active agents that modulate TRAF6/TRAF6-inhibiting protein complex formation. Specifically, leader compounds that either inhibit the formulation of TRAF6/TRAF6-inhibiting protein complexes or prevent or inhibit the dissociation of TRAF6/TRAF6-inhibiting protein complex already formed can be identified by a conventional method. In such aspect, the present invention provides a method for screening a molecule regulating activity of TRAF6-inhibiting proteins or TRAF6, particularly a method for screening a molecule that modulates TRAF6/TRAF6-inhibiting protein complex formation. Preferably, the screened molecule prevents or inhibits the dissociation of TRAF6/TRAF6 inhibiting protein complexes, or inhibits the formation of TRAF6/TRAF6-inhibiting protein complexes. The method comprises culturing a mixture of TRAF6 and TRAF6-inhibiting proteins with a candidate molecule, and detecting the ability of said candidate molecule to modulate the TRAF6/TRAF6-inhibiting protein complex formation, for example to inhibit the interactions between TRAF6 and TRAF6-inhibiting protein, or to prevent or inhibit the dissociation of the TRAF6/TRAF6-inhibiting protein complex.

Molecules capable of preventing the interaction between TRAF6-inhibiting protein and TRAF6 may find utility under conditions where the immune system needs to be strengthened. Agents inhibiting the dissociation of TRAF6/TRAF6-inhibiting protein complexes can be useful as immunosuppressants or anti-inflammatory agents. Screening assays can also be designed to find leader compounds that mimic the biological activity of a native ligand of a TNF receptor superfamily member with which a TRAF6 protein is associated, e.g. TNF, CD40 ligand, etc. These screening methods involve assaying the candidate molecules for their ability to release TRAF6 from inhibition of the TRAF6-inhibiting proteins.

The assays can be performed in a variety of ways, including protein-protein binding assays, biochemical screening assays, immunoassays, cell based assays, etc. Such assay formats are well known in the art.

The assay mixture typically contains one of TRAF6-inhibiting proteins and a TRAF6 protein with which the TRAF6-inhibiting proteins are normally associated, usually in an isolated, partially pure or pure form. One or both of these components may be fused to another peptide or polypeptide, which may, for example, enable or enhance protein-protein binding, or/and improve stability under assay conditions, etc. In addition, TRAF6-inhibiting proteins and/or TRAF6 usually comprise or are coupled to a detectable label. The label may provide for direct detection by measuring radioactivity, luminescence, fluorescence, absorbance, etc., or indirect detection such as an epitope tag, an enzyme, etc. The assay mixture additionally comprises a candidate molecule (a pharmacological agent), and optionally a variety of other components, such as buffers, carrier proteins, e.g. albumin, detergents, protease inhibitors, salts, nuclease inhibitors, antimicrobial agents, etc., which facilitate binding, increase stability, reduce non-specific or background interactions, or otherwise improve the efficiency or sensitivity of the assay.

To screen for inhibitors of TRAF6-inhibiting protein/TRAF6 binding, the assay mixture is incubated under conditions whereby, but for the presence of the candidate molecule, the TRAF6-inhibiting proteins specifically bind the TRAF6 protein with a reference binding affinity. The mixture components can be added in any order as long as the requisite binding can be achieved. Incubation may be performed at any temperature which facilitates optimal binding, typically between about 4° C. and 40° C., more commonly between about 15° C. and 40° C. Incubation periods are selected for optimal binding but are also minimized to facilitate rapid, high-throughput screening. Typically, they are between about 0.1 and 10 hours, preferably less than 5 hours, more preferably less than 2 hours. After incubation, the effect of the candidate molecule on the TRAF6/TRAF6-inhibiting protein binding is determined in any conventional way. For cell-free binding-type assays, a separation step is often used to separate bound and unbound components. Separation may, for example, be effected by precipitation (e.g. TCA precipitation, immunoprecipitation, etc.), immobilization (e.g. on a solid substrate), followed by washing. The bound protein is conveniently detected by taking advantage of a detectable label attached to it, e.g. by measuring radioactive emission, absorbance, etc., or by indirect detection using, e.g. antibody conjugates.

Compounds which inhibit or prevent the dissociation of the TRAF6/TRAF6-inhibiting protein complexes can be conveniently identified by forming the TRAF6/TRAF6-inhibiting protein complex in the absence of the candidate compound, adding the candidate compound to the mixture, and changing the conditions such that, but for the presence of the candidate compound, TRAF6 would be released from the complex. This can, for example, be achieved by changing the incubation temperature or by adding to the mixture a compound which, in the absence of the candidate compound, would release TRAF6 from its complexed form.

In order to identify leader compounds for therapeutically active agents that mimic the biological activity of a native ligand of a TNF receptor superfamily member with which a TRAF6 protein is associated (e.g. TNF, CD40 ligand, etc.), the candidate compound is added to a mixture of TRAF6-inhibiting proteins and TRAF6. The ratio of TRAF6 to TRAF6-inhibiting proteins and the incubation conditions are selected such that TRAF6tRAF6-inhibiting protein complexes are formed prior to the addition of the candidate compound. Upon addition of a candidate compound, its ability to release TRAF6 from the TRAF6/TRAF6-inhibiting protein complex is tested. The typical assay conditions, e.g. incubation temperature, time, separation and detection of bound and unbound material, etc. are as described hereinabove. Upon addition of the candidate compound, its ability to initiate a TRAF6-mediated signaling event is detected. As an end point, it is possible to measure the ability of a candidate compound to induce TRAF6-mediated NF-κB activation m a conventional manner. According to a preferred method, an NF-κB-dependent reporter gene, such as an E-selectin-luciferase reporter construct (Schindler and Baichwal, Mol. Cell. Biol. 14, 5820 (1994)), is used in a cell type assay. Luciferase activities are determined and normalized based on β-galactosidase expression Alteratively, NF-κB activation can be analyzed by electrophoretic mobility shift assay (Schutze et al, Cell 71, 765-776 (1992)). However, other conventional biochemical assays are equally suitable for detecting the release of TRAF6 from its complexed form.

The TRAF6-inhibiting proteins of the present invention can be used to produce antagonist or against anti-TRAF6-inhibiting protein antibodies that block or mimic the ability of TRAF6-inhibiting protein to inhibit TRAF6-mediated signal transduction. Polyclonal or monoclonal antibodies against the TRAF-6 inhibiting proteins can be readily prepared by a known technique (Monoclonal Antibodies: Principles and Practice, Second Ed., James W. Coding, Academic Press (1986)). Antibodies against the TRAF6-inhibiting proteins, fragments of the antibodies (for example, Fab fragments), humanized antibodies, antibodies conjugated to cytotoxin, etc. are included within the scope of the antibodies of the present invention.

The monoclonal antibodies against the TRAF6-inhibiting proteins of the present invention can be obtained by the following method in a particular embodiment. That is, as an antigen for imnunization, native TRAF6-inhibiting proteins or gene recombinant TRAF6-inhibiting proteins can be used Lymphocytes from a mammal which has been injected with respective antigens, or lymphocytes immunized by an in vitro method, are fused with a myeloma cell line, thus producing hybridomas according to a conventional method. The hybridoma culture media was then treated with respective highly purified antigens to screen a hybridoma that produces an antibody capable of recognizing the respective antigen by solid phase ELISA. The obtained hybridomas are cloned to produce a stable antibody-producing hybridoma, which is incubated to obtain a desired antibody. In order to produce a hybridoma, a mouse and rat can be used.

Immunization can be commonly effected by diluting an antigen in an appropriate solvent, for example physiological saline, at an appropriate concentration, transvenously or intraperitoneally administering the resulting solution, in combination with Freund's complete adjuvant, to an animal, in which the adminstration is performed 3 to 4 times at intervals of 1 to 2 weeks. The animal thus immunized is sacrificed and laparotomized at 3 or 4 days after the final immunization, the spleen is harvested and the spleen cells are used as immunized cells. Myeloma cells derived from mouse for cell fusion with the immunized cells include, for example, p3/x63-Ag8, p3-U1, NS-1, MPC-11, SP-2/0, OF, P3x63 Ag 8.653 and S194. Rat derived R-210 cells may also be used Human B lymphocytes are immunized in vitro, and are fused with human myeloma cells or transformed human B lymphocytes with EB virus to produce human type antibody.

Cell-fusion of the immunized cells with the myeloma cell line can be carried out principally by any known method. For example, a method of Koehler and Milstein is generally used (Koehler et al., Nature 256, 495497, 1975) but an electric pulse method using electric pulses can also be applied. The immunized lymphocytes and myeloma cells are mixed at conventional ratios and a FCS-free cell culture medium containing polyethylene glycol is generally used for cell fusion. The fused cells are cultured in HAT selection medium containing FCS to screen fused cells(hybridomas). For screening of hybridomas producing antibody, ELISA, plaque assay, Ouchterlony or agglutination assay can be principally adopted The stable hybridoma thus established can be subcultured by conventional methods and stored underliquid nitrogen, if required. The hybridoma is cultured by known methods or the hybridoma is transplanted to abdominal cavity of mammals and culture broth is recovered as ascite fluid Monoclonal antibody in the recovered cultured broth (or ascite) can be purified by conventional methods such as salting out, ion exchange, and gel filtration, protein A or G affinity chromatography. The resultant antibody can specifically recognize native TRAF6-inhibiting proteins and also functional derivatives, mutants and fragments thereof. The antibody can be measured by assay systems known as radioimmunoassay (RIA) or enzyme immunoassay (EIA) using radioactive isotope-labeling or enzyme-labeling.

In a particular embodiment of the present invention, a polyclonal antibody recognizing the TRAF6-inhibiting proteins can be produced as follows. Host cells expressing TRAF6-inhibiting proteins are pulverized and purified by OCIF immobilized column and gel-filtration chromatography to obtain native TRAF6-inhibiting proteins which is used as an antigen for immunization. Also, a gene recombinant TRAF6-inhibiting protein can be obtained by inserting TRAF6-inhibiting protein cDNA into an expression vector by a conventional method and expressing the resulting recombinant plasmid in animal cells such as CHO cells, BHK cells, Namalwa, COS-7 cells, insect cells or bacillus, followed by purifying the antigen as described above and using it as an antigen for immunization. Alternatively, it is possible to obtain an immunogen by adding a base sequence encoding a known signal sequence derived from another secretory protein upstream of the 5′ terminal of TRAF6-inhibiting protein cDNA, inserting the resulting plasmid into an expression vector by a similar genetic engineering method, expressing the vector in host cells such as various animal cells, insect cells or bacillus, etc., and purifying the expressed protein The immunogen thus obtained is diluted in phosphate buffered saline (PBS), emulsified with an equal amount of Freund's complete adjuvant, if necessary, and subcutaneously administered to the animals several times at an interval of about 1 week to immunize the animals. The antibody titer is measured and an additional administration is performed at a point reaching a maximum antibody titer. At 10 days after the final administration, blood is collected from all the animals. Antisera from the collected blood is fraction precipitated with ammonium sulphate, followed by either purification of the globulin fraction by anion exchange chromatography, or dilution with an equal volume of anti-serum binding buffer (BioRad) and purification of the diluted anti-serum by protein A or protein G sepharose column chromatography, to obtain anti-TRAF6-inhibiting protein polyclonal antibody.

Also, the present invention provides antisense olignucleotides inhibiting the expression of TRAF6-inhibiting proteins. Antisense technology can be used to control gene expression through triple-helix formation or antisense DNA or RNA. Regarding the antisense technology, reference can be made to Okano, J. Neurochem. 56:560 (1991); “Oligodeoxynucleotides as Antisense Inhibitors of Gene Expression.” CRC Press, Boca Raton, Fl. (1988). Regarding the triple helix, reference can be made to Lee et al., Nucl. Acids Res., 6:3073 (1979); Cooney et al, Science, 241:456 (1988); and Dervan et al., Science, 251:1360 (1991). This method is based on binding of polynucleotides to complementary DNA or RNA. For example, the 5′ coding portion of the polynucleotide, which encodes the polypeptide domain of the present invention, is used to design an antisense RNA oligonucleotide of from about 10 to 40 base pairs in length. A DNA oligonucleotide is designed to be complementary to a region of the gene involved in transcription initiation, thereby preventing the transcription and the production of the TRAF6-inhibiting protein. The antisense RNA oligonucleotide hybridizes to the MRNA in vivo and blocks translation of the mRNA molecule into the TRAF6-inhibiting polypeptides. The oligonucleotides described above can also be delivered to cells so that the antisense RNA or DNA may be expressed in vivo to inhibit production of the TRAF6-inhibiting protein.

The antisense nucleic acid of the present invention comprises a complementary sequence to at least a portion of RNA transcripts of the TRAF6-inhibiting protein gene. However, absolute complementarity is not required although it is preferred. “A sequence complementary to at least a portion of RNA”, as referred herein, means a sequence having sufficient complementarity to be capable of hybridizing with the RNA, thereby forming a stable duplex. Therefore, in an embodiment, for double-stranded TRAF6-inhibiting protein antisense nucleic acids, it is possible to test a single strand of the duplex DNA, or assay triplex formation. The ability to hybridize will depend on both the degree of complementarity and the length of the antisense nucleic acid. Generally, the longer the hybridizing nucleic acid, the more base mismatches with a TRAF6-inhibiting protein RNA the nucleic acid may contain but still form a stable duplex (or triplex). One skilled in the art can ascertain a tolerable degree of mismatch by use of standard procedures to determine the melting point of the hybridized complex.

Oligonucleotides that are complementary to the 5′ end of the message, for example, the 5′ untranslated sequence up to and including the AUG initiation codon, should be most effective for inhibiting translation However, sequences complementary to the 3′ untranslated sequences of mRNAs have also shown to be effective to inhibit translation of mRNAs (Wagner, R, 1994, Nature 372:333-335). Therefore, oligonucleotides complementary to either the 5′- or 3′-non-translated, non-coding regions of NSP could be used in an antisense approach to inhibit translation of endogenous TRAF6-inhibiting protein MRNA. Oligonucleotides complementary to the 5′ untranslated region of the MRNA should include the complement of the AUG start codon. Antisense oligonucleotides complementary to mRNA coding regions are less efficient inhibitors of translation but could be used according to the present invention. Whether or not designed to hybridize to the 5′-, 3′- or coding region of TRAF6-inhibiting protein MRNA, antisense nucleic acids should be at least six nucleotides in length, and are preferably oligonucleotides composed of 6 to about 50 nucleotides in length. In specific aspects, the oligonucleotide is composed of at least 10 nucleotides, at least 17 nucleotides, at least 25 nucleotides or at least 50 nucleotides.

The antisense oligonucleotide may comprise at least one modified base moiety selected from a group consisting of 5-fluorouracil, 5-bromouracil, 5-chlorouracil, 5-iodouracil, hypoxanthine, xanthine, 4-acetylcytosine, 5-(carboxyhydroxylmethyl) uracil 5-carboxymethylaminomethyl-2-thiouridine, 5-carboxymethylaminomethyluracil, dihydrouracil, β-D-galactosylquenosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6diaminopurine, but is not limited thereto.

The antisense oligonucleotide may also comprise at least one modified sugar moiety selected from the group consisting of arabinose, 2-fluoroarabinose, xylulose and hexose, but is not limited thereto.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof, but is not limited thereto.

In yet another embodiment, the antisense oligonucleotide is an α-anomeric oligonucleotide. An α-anomeric oligonucleotide forms specific double-stranded hybrids with complementary RNA in which the two strands run parallel to each other, contrary to the usual β-units (Gautier et al., Nucl. Acids Res. 15:6625-6641 (1987)). The oligonucleotide is a 2-O-methylribonucleotide (Inoue et al., Nucl. Acids Res. 15:6131-6148 (1987)), or a chimeric RNA-DNA analogue (Inoue et al., FEBS Lett. 215:327-330 (1997)).

The present invention provides a pharmaceutical composition effective in treating or preventing diseases associated with hyperactivity of TRAF6, NF-κB or AP-1, comprising a therapeutically or prophylactically effective amount of an active ingredient selected from the group consisting of TRAF6-inhibiting protein expression vectors, TRAF6-inhibiting proteins and mRNA thereof, and mixtures of any two or more thereof, alone or in combination with any pharmaceutically acceptable carrier.

In yet another aspect, the present invention provides a method for treating or preventing diseases associated with hyperactivity of TRAF6, NF-κB or AP-1, comprising administering a composition comprising a therapeutically or prophylactically effective amount of an active ingredient selected from the group consisting of TRAF6-inhibiting protein expression vectors, isolated TRAF6-inhibiting proteins and mRNA thereof, and mixtures of any two or more thereof, alone or in combination with any pharmaceutically acceptable carrier, to an animal.

In the present invention, the disease associated with hyperactivity of TRAF6, NF-κ B or AP-1 includes, but is not limited to, osteoporosis, atherosclerosis, asthma, arthritis, cachexia, cancer, diabets, inflammatory bowel diseases, stroke and septic shock (Baldwin AS Jr., J Clin Invest. 2001 107:36; Baldwin AS., J Clin Invest. 2001 107:241-6; Yamamoto Y, Gaynor RB., i Clin Invest. 2001 107:135-42; and Handel ML., Inflamm Res. 1997 46:282-6).

In yet another aspect, the present invention provides a pharmaceutical composition effective in treating or preventing diseases associated with hypoactivity of TRAF6, NF-κB or AP-1, comprising a therapeutically or prophylactically effective amount of an active ingredient selected from the group consisting of antisense RNAs of TRAF6-inhibiting proteins, antibodies against TRAF6-inhibiting proteins and mixtures of any two or more thereof, alone or in combination with any pharmaceutically acceptable carrier.

In yet another aspect, the present invention provides a method for treating or preventing diseases associated with hypoactivity of TRAF6, NF-κB or AP-1, comprising administering a composition comprising a therapeutically or prophylactically effective amount of an active ingredient selected from the group consisting of antisense RNAs of TRAF6-inhibiting proteins, antibodies against TRAF6-inhibiting proteins and mixtures of any two or more thereof, alone or in combination with any pharmaceutically acceptable carrier, to an animal.

In the present invention, the disease associated with hypoactivity of TRAF6, NF-κB or AP-1 includes, but is not limited to, AIDS, Neurodegenerative disorders, autoimmune diseases, immunodeficiencies and stroke (Ballard D. W., Immunol Res 23:157-66, 2001; Hayashi T. et al., Apoptosis 6:31-45, 2001; and Balla A. et al., Biochem. Pharmacol. 61:769-77, 2001).

The TRAF6-inhibiting proteins of the present invention have been found to inhibit generation of osteoclasts. Thus, in yet another aspect, the present invention provides a pharmaceutical composition for treating or preventing metabolic bone diseases comprising an active ingredient selected from a group consisting of TRAF6-inhibiting protein expression plasmids, TRAF6-inhibiting proteins or TRAF6-inhibiting protein MRNA and mixtures of any two or more thereof, alone or in combination with any pharmaceutically acceptable carrier.

In yet another aspect, the present invention provides a method for treating or preventing metabolic bone diseases, comprising administering a composition comprising an active ingredient selected from a group consisting of TRAF6-inhibiting protein expression plasmids, TRAF6-inhibiting proteins or TRAF6-inhibiting protein MRNA and mixtures of any two or more thereof, alone or in combination with any pharmaceutically acceptable carrier, to an animal. Metabolic bone diseases are progressed by collapse of the balance between osteoclasts and osteoblasts.

Representative metabolic bone disease is osteoporosis. Osteoporosis refers to a condition in which proliferation of osteoclasts is increased compared to osteoblasts and consequently, the total bone mass decreases. As osteoporosis is developed, the width of the cortical bone decreases, the medullary cavity expands and reticular bony spicules is lowered, whereby bone becomes porous. As the osteoporosis progresses, physical strength of bone falls, inducing lumbago and arthralgia, and even upon a weak impact, bone tends to be readily broken. Other than osteoporosis, the metabolic bone disease includes lesions on the bone induced by bone metastasis of tumors such as breast carcinoma, prostatic carcinoma, etc., bone cancers of primary type, for example, multiple myeloma, rheumatoid or degenerative arhritis, periodontal disease accompanying destruction of alveolar bone by microorganisms inducing periodontal disease, inflammatory alveolar bone resorption diseases arising after implantation of a dental implant, inflammatory bone resorption disease caused by implantation for fixing bone in the orthopedic surgery field, and Paget's disease, caused by various hereditary factors. Myeloma is a disease by which bone is easily fractured, and accompanied with severe pain, and is developed due to increased activities of osteoclasts by tumor cells. Breast carcinoma or prostatic carcinoma is prone to metastasize to bone and enhance the activity of osteoclasts, thereby destroying bone. In case of rheumatoid arthritis or degenerative arthritis, tumor necrosis factor, Interleukin-1, Interleukin-6 and the like induced by immune response promotes activity of osteoclast, causing local destruction of bone. In case of inflammation caused by infection of organisms inducing periodontal disease, inflammatory cytokines such as tumor necrosis factors, Interleukin-1, Interleulin-6 and the like are also produced as a result of immune response and promotes differentiation of osteoclasts, destroying alveolar bone supporting teeth.

The carriers used in the pharmaceutical composition of the present invention include carriers, adjuvants and vehicles commonly used in the pharmaceutical field and are generally referred to as “pharmaceutically acceptable carriers”. The pharmaceutically acceptable carriers which can be used in the pharmaceutical composition of the present invention include, but are not limited to, ion exchanger, alumina, aluminum stearate, lecithin, serum proteins, for example, human serum albumin, buffer substances such as phosphates, glycine, sorbic acid, potassium sorbate, partial glyceride mixtures of saturated vegetable fatty acids, water, salts or electrolytes, for example protamine sulfate, disodium hydrogen phosphate, potassium hydrogen phosphate, sodium chloride and zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The phannaceutical compositions of the present invention may be administered via any conventional route as long as they can reach a desired tissue. The pharmaceutical composition of the present invention thus may be administered topically, orally, parenterally, intraocularly, transdermally, rectally, intestinally, etc. and formulated into solutions, suspensions, tablets, boluses, capsules, sustained-release formulation and the like. The term “parenteral” as used herein includes subcutaneous, intranasal, intravenous, intraperitoneal, intramuscular, intra-articular, intrasynovial, intrasternal, intracardiac, intrathecal, intralesional and intracranial injection or infusion techniques.

In a preferred embodiment, for parenteral administration, the pharmaceutical composition of the present invention may be prepared in an aqueous solution. Preferably, Hank's solution, Ringer's solution or physically appropriate buffer solution such as physically buffered saline may be used. Aqueous injection suspension may be contain a substance capable of raising viscosity of the suspension, such as sodium carboxymethyl cellulose, sorbitol or dextran. In addition, the suspension of the active ingredient may be prepared as an oily injection suspension in an suitable oil. Suitable liphophilic solvents or carriers include fatty acids such as sesame oil or ethyl oleate, triglyceride or synthetic fatty acid ester such as liposome. Polycationic non-lipid amino polymers also can be used as a vehicle. Optionally, the suspension may contain a suitable stabilizer or agent to increase solubility of the compound and to prepare a solution of high concentration.

The preferred pharmaceutical compositions of the present invention may be in the form of a sterile injectable preparation, for example, as a sterile injectable aqueous or oleaginous suspension. This suspension may be formulated according to techniques known in the art using suitable dispersing or wetting agents, for example, Tween 80, and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the injectable preparations, as are pharnaceutically-acceptable natural oils, for example, olive oil or castor oil, especially in their polyoxyethylated derivatives.

The term “therapeutically effective amount” of the active ingredient as used herein, associated with the composition of the present invention, refers to a dosage level of about 1 mg to about 10 mg/kg body weight per day, typically about 50 mg to about 500 mg/kg body weight/day, for use in treatment of the above described conditions for anmals, particularly human beings. The term “prophylactically effective amount” represents a dosage level of about 0.1 mg to about 1 mg/kg body weight per day, typically about 5 mg to about 50 mg/kg body weight body weight/day, for use in prevention of the above described conditions for animals, particularly human beings.

The dosage for a patient will be determined by a skilled person in the art using techniques known in the art, according to the activity of the used protein or nucleic acid sequence, the age, body weight, general health status, sex, adminstration route, formulation of drug and types of the particular diseases. Generally, in case of the composition comprising polypeptide, the dose of the polypeptide containied in a single dose is about 25 μg to 5 mg per host kg. Suitable dose volume is typically about 0.1 ml to 5 ml though it will be varied according to the size of the patient.

For antibody against TRAF6-inhibiting protein, its amount to be combined with a carrier substance to make a single dose form may be varied according to the host to be treated and specific administration manner, but the preparation to be orally administered to human may contain 50 mg to 500 mg of antibody combined with a pharmaceutically acceptable carrier in an appropriate and convenient amount capable of comprising about 20 to about 40% of the composition. The single dose form generally contains about 10 mg to 50 mg of antibody. Monoclonal antibody in the pharmaceutical composition tends to be attached to glassware such as vials and injection vessels and is unstable. Also, it can easily lose its activity by adverse physical and chemical factors, for example, heat, pH and humidity extremes. Therefore, a stabilizer, pH modifier, buffering agent, solubilizer, surfactant can be added to produce a stable formulation. As the stabilizer, amino acids such as glycine, alanine and the like; sugars such as dextran 40, mannose and the like; sugar alcohol such as sorbitol, mannitol, xyltol and the like can be used and a combination of any two or more thereof can be used. The added amount of these stabilizers is preferably 0.01 to 100 times, particularly 0.1 to 10 times, based on the weight of the antibody. Addition of these stabilizers allows liquid formulation or lyophilized formulation to be improved in storage stability. As the buffering agent, for example, phosphate buffer, citric acid buffer and the like can be used. The buffering agent modifies pH of the aqueous solution upon redissolution of the liquid formulation or lyophiized formulation and is conducive to stability and solubility of the antibody. The added amount of the buffering agent is preferably 1 to 10 mM with respect to the volume after the liquid formulation or lyophilized formulation is redissolved. As the surfactant, polysorbate 20, pluronic F-68, polyethyleneglycol and the like can be used, with polysorbate 80 being preferred, and a combination of any two or more thereof can be used.

As described above, proteins of high molecular weight such as antibodies are prone to attach to glass or resin which is a typical material of the containers used in laboratories. Therefore, a surfactant can be added to prevent the antibody from attaching to a container upon redissolution of liquid formulation or lyopyilized formulation. The added amount of the surfactant is preferably 0.001 to 1.0% with respect to the volume after the liquid formulation or lyophilized formulation is redissolved. Though the formulation of present inventive antibody can be prepared by adding a stabilizer, buffering agent or anti-attaching agent, for an injection formulation for medical or veterinary medicine, acceptable osmosis rate is preferably 1 to 2. The osmosis rate can be adjusted by increasing or decreasing sodium chloride concentration upon formulation. The amount of antibody in the formulation can be suitably adjusted according to diseases to be treated and the employed administration route. The dose of humanized antibody for human depends on affinity of the antibody to human protein NSP, that is, a dissociation constant to human protein NSP. It is possible to exhibit medical effects even when the dose for administration to human is reduced in proportion to the high affinity (low Kd value).

Now, the present invention will be described in detail by the following examples. However, the examples are for illustration of the present invention and do not limit the scope of the present invention thereto.

### EXAMPLE 1

The TRAF6 used as a bait was a gene of human origin and based on GeneBank No. U78798 L81153. Polymerase chain reaction (PCR) was performed using the primer 1 (SEQ ID NO: 1) and primer 2 (SEQ ID NO: 2) to amplify the entire open reading frame (ORF) of full length TRAF6 (amino acids 1-523) and the primer 1 (SEQ ID NO: 1) and the primer 3 (SEQ ID NO: 3) to amplify the N-teminus of TRAF6 (amino acids 1-274). The respective produced bait vectors corresponding to the amplified portion were designated as pGBT9-TRAF6 fill length and pGBT9-TRAF6 dm (Table 1).

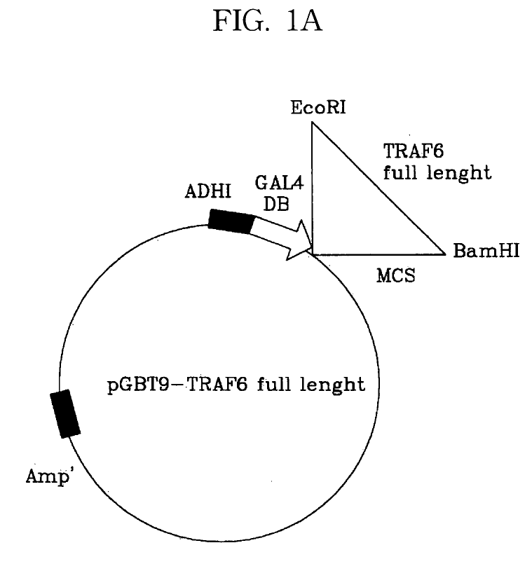

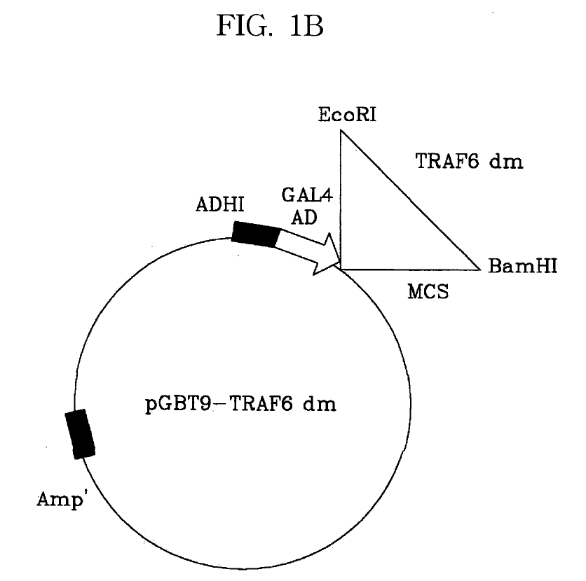

The PCR was performed by repeating 30 cycles of 94° C. (1 minute), 55° C. (1 minute) and 72° C. (1 minute) using Taq polymerase (Gibco BRL). The PCR productes were separated using EcoRI and BamHI and subcloned into the vector pGBT9 (Clontech, Palo Alto, Calif.) containing GALA DNA binding domain, respectively to construct pGBT9-TRAF6 full length and pGBT9-TRAF6 dm. (FIGS. 1A and 1B).

TABLE 1
Primer sequence

| | |
|---|---|
| Promoter for producing | TRAF6-YF 5′-CGGGATTCAGTCTGCTAAACTGTGAA-3′ |
| pGBT9-TRAF6 full length | (Primer 1, Sense: SEQ ID NO: 1) |
| | TRAF6-YR1 5′-CGGGATCCCTATACCCCTGCATCAGT-3′ |
| | (Primer 2, Antisense: SEQ ID NO: 2) |
| Promoter for producing | TRAF6-YF 5′-CGGGATTCAGTCTGCTAAACTGTGAA-3′ |
| pGBT9-TRAF6 dm | (Primer 1, Sense: SEQ ID NO: 1) |
| | TRAF6-YR2 5′-CGGGATCCGGCCAACATTCTCATGTGT-3′ |
| | (Primer 3, Antisense: SEQ ID NO: 3) |
| Promoter for producing | TBZF-F 5′-CGGAATTCGGACTGTTGACATTTAGGGAT-3′ |
| pRX-Flag-TBZF | (Primer 4, Sense: SEQ ID NO: 4) |
| | TBZF-R 5′-ACGCGTCGACTCACACATTCCAGTTCTGTAG-3′ |
| | (Primer 1, Antisense: SEQ ID NO: 5) |

### EXAMPLE 2

### Assessment of Ability of Bait Vector to Activate Basic Transcription of β-galactosidase.

The bait vectors of pGBT9-TRAF6 full length and pGBT9-TRAF6 dim constructed in Example 1 were examined by filter β-galactosidase assays to determine whether the bait vectors maintained the ability to activate transcription. The pGBT9-TRAF6 full length and pGBT9-TRAF6 dm vectors were cotransformed into yeast Y190 (Clontech) by a thermal treatment (42° C., 15 minutes), seeded in medium lacking leucine (Leu) or tryptophan (Trp), and cultured 30° C. The yeast in which the bait vectors are transformed can grow in medium lacking tryptophan. The grown yeasts were transferred to a filter paper, which was then dipped into liquid nitrogen for rapid freezing. The frozen yeasts were then thawed at room temperature to lyse the cell wall. The cell lysate was stained with X-gal (Sigma), a substrate of β-galactosidase and observed for development of blue color (Table 2).

The yeasts either transformed with only pGBT9-TRAF6 full length as a bait vector or cotransformed with pGBT9-TRAF6 fill length as a bait vector and pGAD424 vector (Clontech) as a prey developed blue color and thus it was proven that the pGBT9-TRAF6 full length produces positive clones having β-galactosidase trascription activity. On the other hand, the yeasts either transformed with only pGBT9-TRAF6 dm as a bait vector or co-transformed with pGBT9-TRAF6 dm as a bait vector and pGAD424 vector as a prey did not develop blue color and thus it is proven that the pGBT9-TRAF6 dm produces negative clones not having a β-galactosidase trascription activity. The pGBT9-TRAF6 dm vector was used as a bait vector in the subsequent yeast two-hybrid assay.

TABLE 2
Test of basic activity of TRAF6 bait construct

| Binding domain (BD) in | Transcription activation domain | |
|---|---|---|
| pGBT9 | (AD) in pGAD424 | Interaction |
| TRAF6 full length | | Positive |
| TRAF6 full length | pGAD424 | Positive |
| TRAF6 dm | | Negative |
| TRAF6 dm | pGAD424 | Negative |

### EXAMPLE 3

### Cloning of Novel Zinc Finger Protein Binding to TRAF6 Protein by Yeast Two-hybrid System

The yeast two-hybrid system was used to obtain a protein interacting with TRAF6. The yeast two-hybrid analysis was performed following the method described in MATCHMAKER protocol (Clontech). Firstly, the pGBT9-TRAF6 dm bait vector constructed in Example 1 was transformed into Y190 (Clontech), which was then used to produce competent cells. The competent cells were subsequently transformed by thermal treatment (42° C., 15 minutes) with HeLa cDNA library (prey vector) made of pGADGH (Clontech). The transformed yeasts were cultured in SD medium (0.67% yeast nitrogen base without amino acid, 1.5% agar, 10% 10×dropout solution, 2% dextrose) lacking leucine, tryptophan and histidine (His) and supplemented with 10 mM 3-aminotriazone (3-AT) (Sigma), at 30° C. The yeast transformants were selected based on the histidine prototrophy and β-galactosidase expression. Thus, the yeast which grew rapidly on a medium laking histidine was selected and tested by the filter β-galactosidase assay to obtain 14 positive clones.

A prey vector with an unknown binding gene was isolated from a positive clone showing the strongest viability. The unknown binding gene was found to have a size of about 750 bp by decomposition of various restriction enzymes. The gene was sequenced using an automatic sequencer (ABI PRISM 310 Genetic Analyser) and its cDNA sequence was assayed using GeneBank BLAST homology research program. From the BLAST homology research, the clone was found to be a novel protein which had not been reported. Since the protein possesses a zinc finger region, the present inventors initially named this protein as human “TRAF6 binding zinc finger (TBZF) protein”. However, in the specification of the present application, the protein is referred to as “TRAF6-inhibiting protein”, considering the activity to inhibit the activity of TRAF6 proteins, as described in the following examples.

In order to clone full length TRAF6-inhibiting protein, 5′ RACE (rapid amplification of cDNA 5′-ends) and 3′ RACE PCR (GIBCO BRL) using HUVEC cDNA were performed and TRAF6-inhibiting protein gene of 1757bp was obtained. The gene-specific primer 5′-GTTAACAGCTTTTTCACATTCTTC-3′ and 5′-GGCCAGAGCAGAACCATAAAAGAT-3′ were used for the 5′ RACE and 3′ RACE, respectively. The full length cDNA sequence of TRAF6-inhibiting protein and the amino acid sequence deduced therefrom are shown in FIG. 5 (SEQ ID NO: 6) and FIG. 6a (SEQ ID NO: 7). The TRAF6-inhibiting protein contained two characteristic portions of a KRAB domain (amino acid 1-63) and Cys2-His3 type zinc finger (C2H2) domain (FIG. 6A) and exhibites homology with human zinc finger protein 85 (FIGS. 6B and 6C).

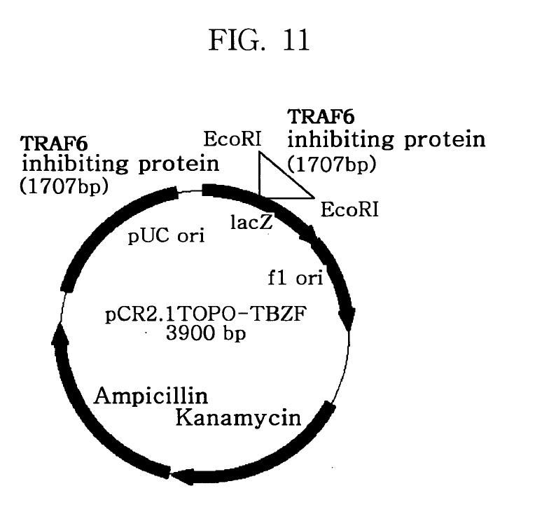

The full length cDNA of the TRAF6-inhibiting protein obtained as described above was subcloned into pCR2.1TOPO-TBZF vector (hivitrogen) to produce a recombinant plasmid expressing the TRAF6-inhibiting protein. The recombinant plasmid was designated as pCR2.1TOPO-TBZF and deposited in Korean Collection for Type Cultures which is an international depository authority under the regulations of Budapest Treaty on the International Recognition of the Deposit of Micro-organisms for the Purpose of Patent Procedures, on Feb. 16, 2001 as a Deposition Access No. KCTC-0963BP. Also, the gene map of the recombinant plasmid pCR2.1TOPO-TBZF of the present invention is shown in FIG. 11 for reference.

The full length cDNA (1757 bp) of the TRAF6-inhibiting protein was cloned into EcoRI-BamHI sites of OKpGAD424 vector to constuct an expression vector expressing TRAF6-inhibiting protein. The vector was then cloned into the yeast Y190 by a thermal impact method to express recombinant TRAF6-inhibiting protein fused with the trascription activation domain of GAL4 in the yeast.

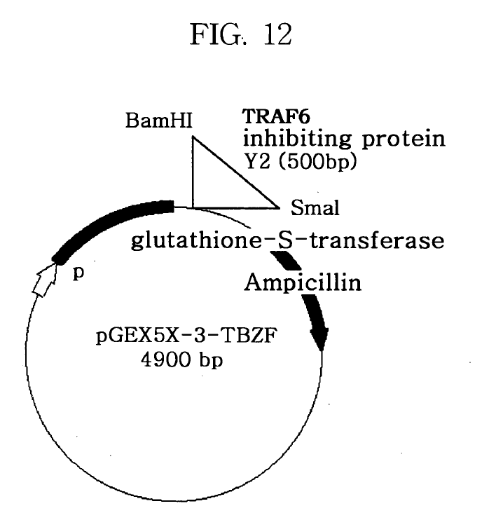

Furthermore, a cDNA segment of about 50 bp was obtained by PCR amplification using the recombinant plasmid pCR2.1TOPO-TBZF as a template. For this PCR amplification, primers of 5′-CGGGATCCCGCATAAGATAAAACATAT-3′ and 5′-AACTGCAGTCACACATTCCAGTTCTG-3′ were used. The primers were cut with the restriction enzyme PstI and the ends were blunt ended. Then, the products were cut with the restriction enzyme BamHI and cloned into GST vector pGEX5X-3 (Pharmacia) cut with the restriction enzymes BamHI and SmaI. The resulting recombinant plasmid was designated as pGEX5X-3-TBZF and its gene map is shown in FIG. 12.

### EXAMPLE 4

### Verification of Interaction of TRAF6-inhibiting Protein with TRAF6 by Yeast Two-hybrid System

In order to confirm the binding specificity of the TRAF6-inhibiting protein, the interactions of the recombinant TRAF6-inhibiting protein of the present invention obtained from Example 3 with TRAF2 and TRAF3, belonging to TRAF superfamily, and a LRRY 1, which is a non-TRAF superfamily protein, were examined.

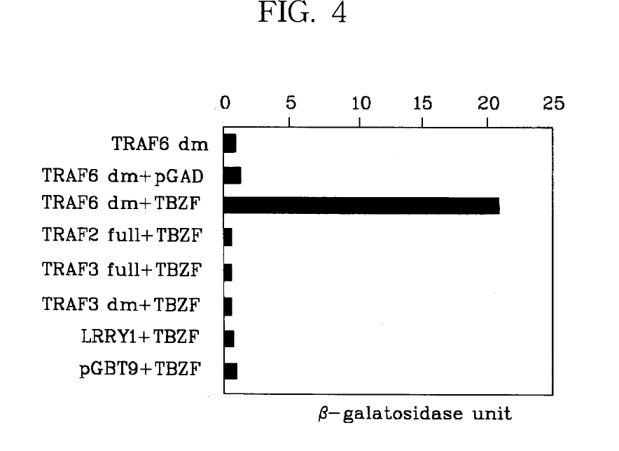

Liquid β-galactosidase assays were performed using O-nitro-phenyl-D-galactopyranoside (Sigma) as a substrate (FIG. 4). When either only TRAF6 dm was expressed or only TRAF6 and transcription activation domain (AD) of GAL4 were expressed, the β-galactosidase activity titers were background level, while the β-galactosidase activity titer was 21 unit upon co-expression of TBZF and TRAF6 with C-terminus deleted (TRAF6 dm). Also, when the TRAF6-inhibiting protein was expressed with TRAF 2 (amino acid 1-501), TRAF 3 (amino acid 1-569), TRAF 3 dm (amino acid 1-268), LRRY1 or DNA binding domain of GAL4 (pGBT9 vector), the β-galactosidase activity titers were very low. From these results, it was demonstrated that there was a specific interaction between TRAF6-inhibiting protein and TRAF6 dm. The binding specificity of the TRAF6-inhibiting protein shown in FIG. 4 was confirmed by the results of filter β-galactosidase assays (Table 3).

TABLE 3
Interaction of TRAF6 dm (amino acid 1-274)
with TRAF6-inhibiting protein in yeast

| Bait vector (pGBT9) | Prey vector (pGAD424) | Interaction |
|---|---|---|
| TRAF6 dm | | Negative |
| TRAF6 dm | PGAD424 | Negative |
| TRAF6 dm | TRAF6-inhibiting protein | Positive |
| Full length TRAF 2 | TRAF6-inhibiting protein | Negative |
| Full length TRAF 3 | TRAF6-inhibiting protein | Negative |
| TRAF3 dm | TRAF6-inhibiting protein | Negative |
| LRRY 1 | TRAF6-inhibiting protein | Negative |
| PGBT9 | TRAF6-inhibiting protein | Negative |

### EXAMPLE 5

### Blast Cellular Differentiation of TRAF6 and TRAF6 Inhibitoy Protein in 293T Cell

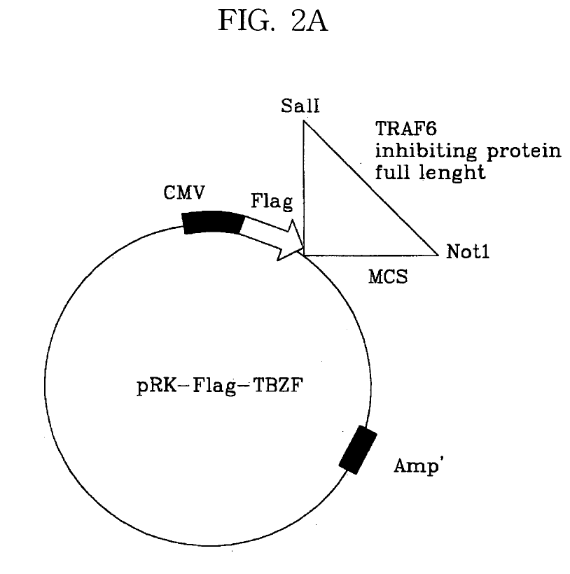

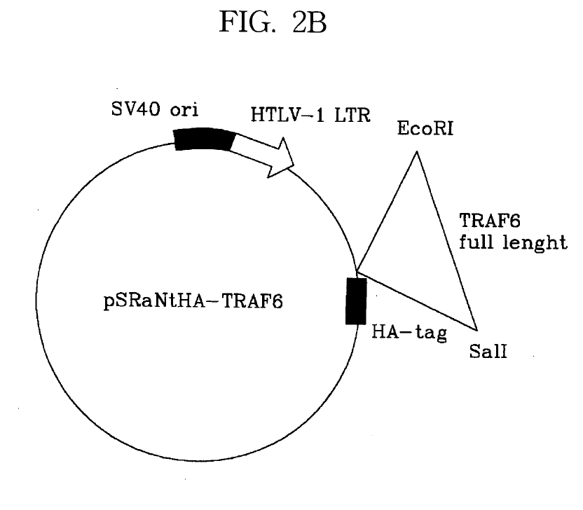

The specific interaction of TRAF6-inhibiting protein with TRAF6 was confirmed through the yeast two-hybrid system and thus, full length TRAF6-inhibiting protein cDNA was amplified using Primer 4 and Primer 5 (Table 1) to determine if the TRAF6-inhibiting protein is located at the same blast cellular position as the TRAF6. The amplified sequences were inserted into pRK-Flag, a mammalian expression vector by which a Flag could be tagged at a N-terminus of the TRAF6-inhibiting protein, producing the pRK-Flag-TBZF vector (FIG. 2A). Also, the amplified sequences were inserted into pSR a NtHA, a mammalian cell expression vector by which HA (hemagglutinin) could be tagged at N-terminus of the TRAF6-inhibiting protein, producing the pSR α NtHA-TBZF vector (FIG. 2B).

The pRK-Flag-TBZF vector (FIG. 2A) was transfected into 293T cells, a mammalian cell line, by calcium phosphate precipitation. 2 days after the transfection, the cells were washed with cold TMNS buffer solution (10 mM Tris-Cl, pH 6.8, 100 mM NaCl, 3 mM MgC₂, 300 mM sucrose), and re-suspended in cold TMNS with protease inhibitors and phosphatase added. The suspension of the transfected cells were centrifuged (600×g, 4° C., 5 minutes) to precipitate nuclei. The supernatant was again centried (100,000×g, 4° C., 75 minutes). The resulting supernatant comprised soluble proteins and the resulting pellet comprised membrane matrix or insoluble substances (Berezney and Coffey, Biochem. Biophys. Res. Commnun., 60, 1410-1417, 1974). The 600×g pellets were re-suspended in buffer A (10 mM Hepes, pH 7.9, 10 mM KCl, 300 mM sucrose, 1.5 mM MgC₂, 0.5 mM DTT, 0.5% NP-40), with a protease inhibitor added. The suspension was centrifuged (14,000 rpm, 4° C., 10 seconds). The resulting pellets were washed with the buffer A and re-suspended in a buffer B (20 mM Hepes, pH 7.9, 20% glycerol, 100 mM KCl, 100 mM NaCl, 0.2 mM EDTA, 0.5 mM PMSF, 0.5 mM DTT) with a protease inhibitor added. The nuclear extract was frozen with liquid nitrogen, thawed over ice, sonicated, and centrifuged (14,000 rpm, 4° C., 30 minutes). The nuclei were fractionated into an insoluble nuclear matrix and a soluble chromatin-containing fraction.

The respective fractionated samples were analysed by SDS-PAGE, or transferred onto a membrane for a Western blot. In the Western blot, as a primary antibody, anti-TRAF6 antibody (Santa Cruz Biotechnology), anti-Flag antibody and anti-tubulin antibody (Cedarlane) were used for TRAF6, TRAF6-inhibiting protein and tubulin present in the insoluble fraction, respectively (FIG. 7). The fractions shown in FIG. 7 are as follows:

(N): nucleus

1'S soluble chromatin-containing fractions;

2'S: soluble nuclear protein fractions;

P: insoluble nuclear matrix;

(C): cytoplasm

S: soluble proteins:

P: membrane matrix or insoluble proteins.

TRAF6 was found in both nucleus (N) and cytoplasm (C) while the TRAF6-inhibiting protein was not detected in insoluble fractions of cytoplasm (100,000×g sup(s)) (FIG. 7, lanes 15 and 16). From these results, it can be seen that the TRAF6 and TRAF6-inhibiting protein co-exist in the nuclear fractions and insoluble cytoplasmic fractions (FIG. 7).

### EXAMPLE 6

### Verification of Intracelluar Distribution of TRAF6 and TRAF6-inhibiting Protein by Confocal Microscopy

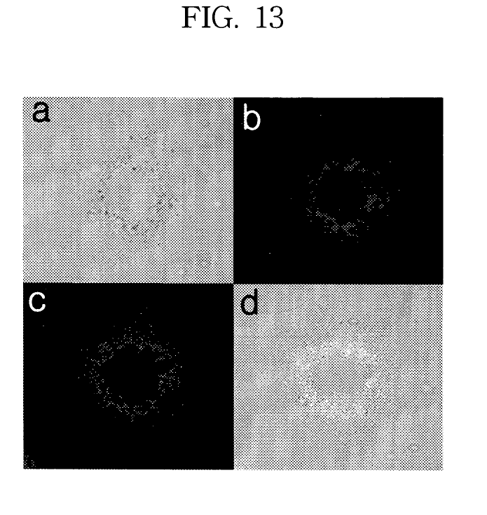

In order to determine the distribution of TRAF6 and TRAF6-inhibiting protein in the cell, full length cDNAs of TRAF6-inhibiting protein and TRAF6 were subcloned into pDsRed1-C1 and pEGFP-C2 to produce recombinant plasmids designated as pDsRed-C1-TBZF and pEGEP-C2-TRAF6, respectively. The used vectors were commercially obtained from Clontech. Following the instruction of the manufacturer, COS-7 cell line was transfected with pDsRed-Cl-TBZF and pEGFP-C2-TRAF6 using Superfect (Qiagen). 24 hours after transfection, the respective transfectants were subjected to confocal microscopy using Carl Zeiss Axiovert 135M microscope and LSM410 at Korea Basic Science Institute (Daechon, Korea). The TRAF6 and TRAF6-inhibiting protein were expressed as RFP and GFP fusion proteins. As shown in FIG. 13, both the TRAF6 and TRAF6-inhibiting protein were detected in perinuclear cytoplasmic regions as dot-like structures. Also, weak signals of TRAF6-inhibiting protein were observed in the nucleus (FIG. 13). The red and green channels were superimposed to reveal yellow signals in many locations, but not all. This indicates that TRAF6 and TRAF6-inhibiting protein co-exist in separate intracellular regions (FIG. 13D).

### Example 7

### Interaction of TRAF6 with TRAF6-inhibiting Protein in Mammalian Cells

In order to determine whether TRAF6-inhibiting protein interacts with TRAF6 in not only yeast but also mammalian cells, coimmunoprecipitation was performed. PRK-Flag-TBZF and pSRαNtHA-TRAF6 vectors were transfected into 293t cells by calcium phosphate precipitation following the method described for Example 5. 2 days after transfection, cells were collected and extracted following the method described for Example 5. The resulting cell extracts were incubated with 2 μl of anti-Flag antibody for 1 hour and with 25 μl of protein A agaros (PERCE) for 1 hour. The immunoprecipitations were washed 5 times with a buffer solution B used in Example 5. Coprecipitated proteins were separated by SDS-PAGE and a Westem blot assay was performed using anti-TRAF6 antibody (Santa Cruz Biotechnology) (FIG. 8A).

As shown in FIG. 8A, TRAF6 and TRAF6-inhibiting protein were co-precipitated. This indicates that the anti-Flag antibody recognized TRAF6-inhibiting protein, TRAF6-inhibiting protein interacted with TRAF6, and the TRAF6 was recognized by the anti-TRAF6 antibody, suggesting that TRAF6-inhibiting protein interacts with TRAF6 in animal cells.

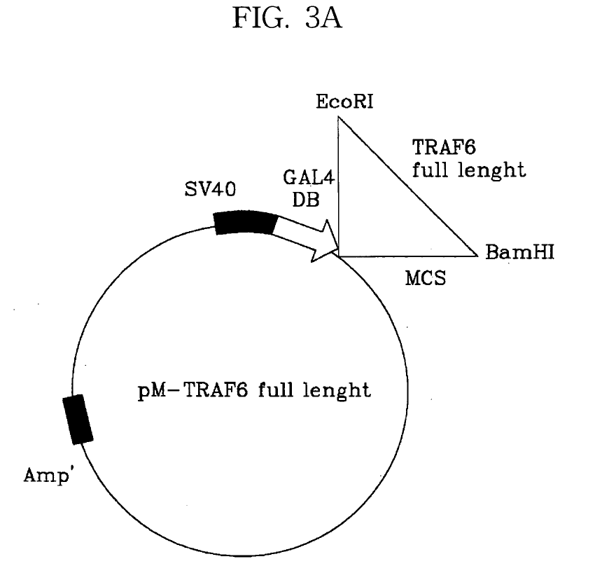

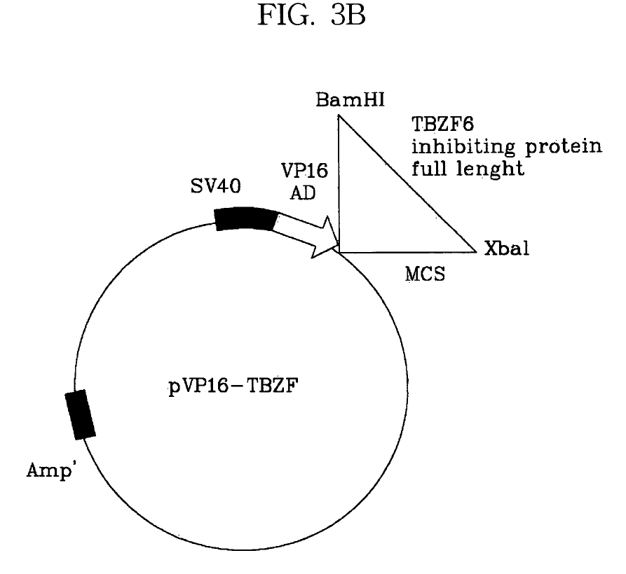

To examine the interaction of TRAF6-inhibiting protein with TRAF6, mammalian two-hybrid assays were also performed. The full length cDNA of TRAF6 was subcloned into the pM vector containing GALA DNA binding domain (DB) to construct pM-TRAF6 fill length (FIG. 3A). The fill length cDNA of TRAF6-inhibiting protein was subcloned into the pVP16 plasmid containing the VP16 transcription activation domain to construct pVP16-TBZF (FIG. 3B). The used expression vector pM, pVP16 and Gal4/Tx-luc construct were supplied by Professor Lee Jae Un at Chonnam University (Korea).

The mammalian two-hybrid assays were performed following the method described in MAT body weight CHMAKER protocol. 293/ENA (Invitrogen) cells were transiently transfected with expression vectors of pM-TRAF6 full length (FIG. 3A) and pVP16-TBZF (FIG. 3B) encoding the GAL4 DNA binding domain or VP16 activation domain, and GAL4/Tx-luc reporter gene. 24 hours later, the cells were lysed with 150 μl of Reporter lysis buffer solution (Promega). 20 μl of the lysate was mixed with 50 μl of luciferase analysis indicator (Luciferase analysis kit, Promega). The luciferase activity was measured with a luminometer. The results are shown in FIG. 8B. The numbers shown on the vertical axis are normalized luciferase activities relative to the control and show mean values (±SD) of triplicate samples.

When the cells were transfected with TRAF6-fusion protein(FIG. 8B, middle bar), weak reporter gene activation was observed. The cells in which TRAF6 and TRAF6-inhibiting protein were co-expressed showed an increased activity (FIG. 8B, the third bar). These results confirm that TRAF6 interacts with TBZF in mammalian cells.

### EXAMPLE 8

### Inhibition of TRAF6-mediated NF-κB and AP- 1 Activation by TRAF6-inhibiting Protein

Over-expression of TRAF6 causes strong activation of nuclear transcription factors NF-κB and AP-1. Therefore, in this example, effects of the expression of TRAF6-inhibiting protein on TRAF6-mediated activation of NF-κB and AP-1 was determined by a luciferase activity assay.

293/EBNA (Invitrogen) cells (1×10⁵) were transfected using Superfect (QIAGEN) in a 24-well plate. To the transfected reactant of each well 50 ng of NF-κB-luc or 50 ng of AP-1-luc were added with an expression vector expressing TRAF6-inhibiting protein. 24 hours later, the cells were lysed with 150 μl of the buffer solution used in Example 6. 20 μl of the lysate was mixed with 50 μl of the luciferase analysis indicator and measured for luciferase activity. The results are shown in FIG. 9A (NF-κB) and FIG. 9B (AP- 1). The numbers shown on the y-axis are normalized luciferase activities relative to the control and show mean values (±SD) of triplicate samples.

Over-expression of TRAF6 in 293/EBNA cells resulted in strong activation of the NF-κB responsive reporter gene. Co-expression of TRAF6-inhibiting protein inhibited the TRAF6-mediated NF-κB activation in a dose-dependent manner (FIG. 9A). Also, over-expression of TRAF6 in 293/EBNA activated the AP-1 responsive reporter gene and over-expression of TRAF6-inhibiting protein blocked the TRAF6-mediated AP-1 activation in a dose-dependent manner (FIG. 9B).

It is known that the NF-κB activation by RANK and IL-1R is mediated by TRAF6. Therefore, whether TRAF6-inhibiting protein may interfere with the NF-κB activation upon over-expression of RANK and IL-1R was examined by the above-described luciferase activity assay. As a result, it was shown that TRAF6-inhibiting protein strongly inhibited the RANK-mediated NF-κB activation (FIG. 9C) and weakly inhibited the IL-1R-mediated NF-κB activation (FIG. 9D).

In conclusion, it was noted that TRAF6-inhibiting protein suppressed the NF-κB and AP-1 activation mediated by TRAF6 and the NF-κB activation mediated by RANK and IL-1R (FIGS. 9A to 9D). TRAF6 was shown to involve RANK and IL-1R in the NF-κB activation process and also in the NF-κB by CD40, IL-17, L-18 and other TNFR superfamily proteins(Bocker et al., J.Biol. Chem. 275, 12207-12213, 2000; Kashiwada et al., J. Exp. Med., 187, 237-244, 1998; and Schwandnet et al., J. Exp. Med., 191, 1233-1240, 2000). Therefore, the binding of TRAF6-inhibiting protein to TRAF6 may provide a means of suppressing NF-κB activation by various members of the TNF and IL-1 receptor superfamily.

### EXAMPLE 9

### Inhibition of TNF-mediated and IL-1β-mediated NF-κB Activation by TRAF6-inhibiting Protein

Effect of TRAF6-inhibiting protein on the TNF-mediated NF-κB activation was examined by the luciferase activity assay as described in Example 7. As seen from FIG. 10A, treatment of 293/EBNA (Invitrogen) cells with TNF led to an increase in the NF-κB reporter gene activity (FIG. 10A, white bar). However, upon co-expression of TRAF6-inhibiting protein, the TNF-mediated NF-κB activation was considerably reduced.

Furthermore, the present inventors investigated the effect of TRAF6-inhibiting protein on the IL-1β-mediated NF-κB activation as described above. IL-1β was known to activate NF-κB through a route different from TNF (Cao et al., Nature, 383, 443-446, 1996). IL-1β treatment induced NF-κB activation but co-expression of TRAF6-inhibiting protein considerably reduced the NF-κB activity induced by IL-1β, even lower than that by TNF (FIG. 10B). These results demonstrate that TRAF6-inhibiting protein can by used as an inhibitor of NF-κB activation induced by different stimuli.

### EXAMPLE 10

### Inhibition of Differentiation of Osteoclasts by TRAF6-inhibiting Protein

The effect of the TRAF6-inhibiting protein on differentiation of osteoclasts were tested as follows. Raw264.7 cells which can be differentiated into osteoclasts by incubation for 4 to days with RANKL (receptor activating hgand of NF-κB) was suspended in DMEM/10% FBS, seeded at 4×10⁴/well in 24-well plates, and cultured for 24 hours. The cells were then incubated with the mixture of 1 μg of control vector DNA or TRAF6-inhibiting protein expression plasmid pCR2.1TOPO-TBZF, Tfx™-50 reagent (Promega) and 200 μl of serum-free medium. The medium was changed to α-MEM/10% FBS containing 50 ng/ml RANKL. After 3 days of culturing, the medium was replenished and the culturing was continued. The next day, the cells were washed and stained for tartrate-resistant acid phophatase (TRAP) as a marker of osteoclasts using the Leukocyte Acid Phosphatase Assay Kit (Sigma).

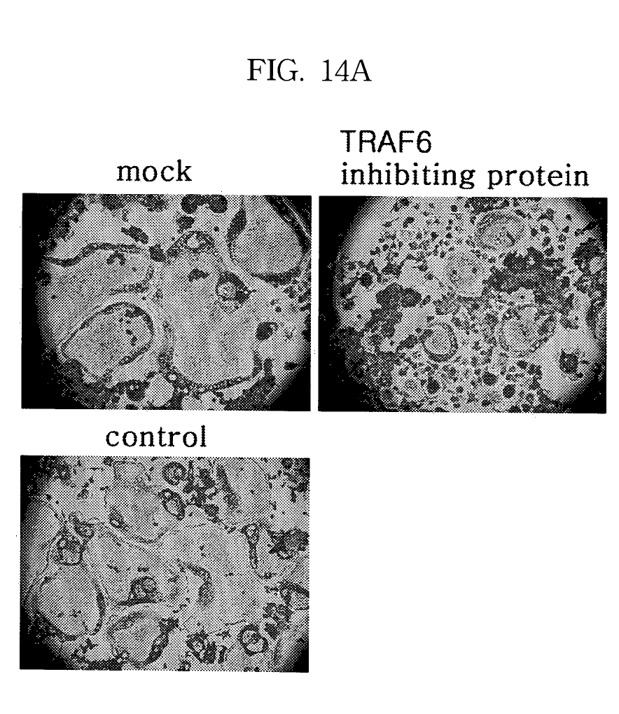

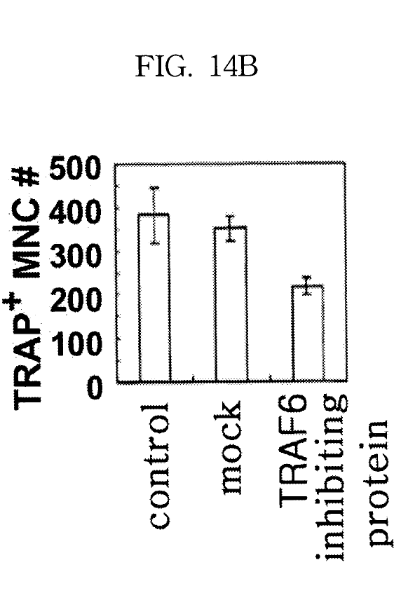

As shown in FIG. 14A, transfection by TRAF6-inhibiting protein expression plasmid significantly reduced the number of TRAP-positive osteoclasts, compared to transfection by control vector. Mock transfection showed little difference compared to non-transfected control cells. This suggests that the transfection method used did not affect the differentiation of Raw264.7 cells under the experimental conditions. Quantitation of TRAP-positive osteoclasts revealed about 40% reduction in the number of osteoclasts containing more than 10 nuclei upon transfection with TRAF6-inhibiting protein expression plasmid (FIG. 14B). These results suggest that it is important to properly regulate expression of TRAF6-inhibiting proteins for osteoclast differentiation.

What is claimed is:

1. An isolated TRAF6-inhibiting protein comprising a KRAB domain at its N-terminus and fourteen C2H2 type zinc finger motifs at its C-terminus, which binds to TRAF6 but does not bind to TRAF2 and TRAF3, and inhibits activity of TRAF6, NF-κB or AP-1.

2. The isolated TRAF6-inhibiting protein of claim 1, wherein the TRAF6-inhibiting protein is derived from human.

3. The isolated TRAF6-inhibiting protein of claim 1, wherein the TRAF6-inhibiting protein comprises the sequence described in SEQ ID NO: 7.

4. A nucleic acid sequence encoding the TRAF6-inhibiting protein defined in any of claims 1 to 3.

5. The nucleic acid sequence of claim 4, wherein the nucleic acid sequence comprises gDNA, cDAN or RNA.

6. A nucleic acid sequence comprising the sequence described in SEQ ID NO: 6.

7. A vector comprising a nucleic acid sequence encoding the TRAF6-inhibiting protein defined in claim 1.

8. The vector of claim 7, wherein the nucleic acid sequence comprises the sequence described in SEQ ID NO: 6.

9. A host cell or cell line transformed or transfected with the vector defined in claims 7 or 8.

10. The transfected host cell or cell line of claim 9, wherein the host cell is yeast, 293T cell or 293/EBNA.

11. A method for producing isolated TRAF6-inhibiting proteins comprising culturing the transformed or transfected host cell or cell line of claim 9 and purifying TRAF6-inhibiting proteins from the culture.

12. A method for purifying a molecule capable of selectively binding to the TRAF6-inhibiting proteins of claim 1 comprising culturing the TRAF6-inhibiting protein with a candidate molecule, isolating the resulting complex, and dissociating the TRAF6-inhibiting protein from the isolated complex.

13. The method of claim 12, wherein the candidate molecule is TRAF6.

14. A method for screening a substance capable of modulating activity of a TRAF6-inhibiting protein comprising culturing a mixture containing TRAF6 and TRAF6-inhibiting protein along with a candidate molecule and detecting the ability of the candidate molecule to modulate the formation of the TRAF6/TRAF6-inhibiting protein complex or the ability of the candidate molecule to prevent or inhibit the dissociation of the TRAF6/TRAF6-inhibiting protein complex.

15. An antibody directed against the TRAF6-inhibiting protein of claim 1.

16. A pharmaceutical composition useful in treating or preventing diseases associated with hyperactivity of TRAF6, NF-κB or AP- 1, comprising a therapeutically or prophylactically effective amount of an active ingredient selected from the group consisting of expression vectors of TRAF6-inhibiting proteins, isolated mRNAs of TRAF6-inhibiting proteins, TRAF6-inhibiting proteins, and mixtures of any two or more thereof, alone or in combination with any pharmaceutically acceptable carrier.

17. The pharmaceutical composition of claim 16, wherein the disease is selected from a group consisting of osteoporosis, atherosclerosis, asthma, arthritis, cachexia, cancer, diabets, inflammatory bowel diseases, stroke and septic shock.

18. A method for treating or preventing diseases associated with hyperactivity of TRAF6, NF-κB or AP- 1, comprising administering a composition comprising a therapeutically or prophylactically effective amount of an active ingredient selected from the group consisting of expression vectors of TRAF6-inhibiting proteins, isolated mRNAs of TRAF6inhibiting proteins, TRAF6- biting proteins, and mixtures of any two or more thereof alone or in combination with any pharmaceutically acceptable carrier, to an animal.

19. A phannaceutical composition useful in treating or preventing diseases associated with hypoactivity of TRAF6, NF-κB or AP- 1, comprising a therapeutically or prophylactically effective amount of an active ingredient selected from the group consisting of antisense RNAs of TRAF6-inhibiting proteins, antibodies directed against TRAF6-inhibiting proteins, and mixtures of any two or more thereof, alone or in combination with any pharmaceutically acceptable carrier.

20. The pharmaceutical composition of claim 19, wherein the disease is selected from a group consisting of AIDS, neurodegenerative disorders, autoimmune diseases, immunodeficiencies and stroke.

21. A method for treating or preventing diseases associated with hypoactivity of TRAF6, NF-κB or AP-1, comprising administering a composition comprising a therapeutically or prophylactically effective amount of an active ingredient selected from the group consisting of antisense RNA of TRAF6-inhibiting proteins, antibodies directed against TRAF6-inhibiting proteins, and mixtures of any two or more thereof alone or in combination with any pharmaceutically acceptable carrier, to an animal.

22. A recombinant plasmid deposited as a Deposit Accession No. KCTC-0963BP.

23. A pharmaceutical composition for treating or preventing metabolic bone diseases comprising an active ingredient selected from a group consisting of expression plasmids of TRAF6-inhibiting proteins, isolated mRNAs of TRAF6-inhibiting proteins, TRAF6-inhibiting proteins, and mixtures of any two or more thereof, alone or in combination with any pharmaceutically acceptable carrier.

24. The pharmaceutical composition of claim 23, wherein the metabolic bone disease is selected from a group consisting of osteoporosis, lesions on the bone induced by bone metastasis of tumors, bone cancers of primary type, rheumatoid or degenerative arthritis, periodontal disease, inflammatory alveolar bone resorption diseases arising after implantation of a dental implant inflammatory bone resorption disease caused by implantation for fixing bone and Paget's disease.

25. A method for treating or preventing metabolic bone diseases, comprising administering a composition comprising an active ingredient selected from a group consisting of expression plasmids of TRAF6-inhibiting proteins, isolated mRNAs of TRAF6-inhibiting proteins, TRAF6-inhibiting proteins, and mixtures of any two or more thereof, alone or in combination with any pharmaceutically acceptable carrier, to an animal.


SEQUENCE LISTING (52 sequences)

SEQ ID NO: 1 - DNA, 26 bp, Artificial
  Primer for amplifying entire open reading frame of TRAF6
cgggattcagtctgctaaactgtgaa

SEQ ID NO: 2 - DNA, 26 bp, Artificial
  Primer for amplifying entire open reading frame of TRAF6
cgggatccctatacccctgcatcagt

SEQ ID NO: 3 - DNA, 27 bp, Artificial
  Primer for amplifying N-terminus of TRAF6 (amino acids 1-274)
cgggatccggccaacattctcatgtgt

SEQ ID NO: 4 - DNA, 29 bp, Artificial
  Primer for amplifying ORF of TRAF-6-inhibiting protein
cggaattcggactgttgacatttagggat

SEQ ID NO: 5 - DNA, 31 bp, Artificial
  Primer for amplifying TRAF6-inhibiting protein
acgcgtcgactcacacattccagttctgtag

SEQ ID NO: 6 - DNA, 1757 bp, Homo sapiens
ccaggacccccctggaagcctagaaatgggactgttgacatttagggatgtggccatagaattctctctggaagaatggcaatgcctggacactgcacagcggaatttatgtaaaaatgtgattttagagaactacagaaacctggtcttcctgggtattgctgtctctaagcaagacctgatcacctgtctggagcaagaaaaagagcctttgactgtgaagagacatgagatggtgaatgaacccccagtaatgtgttctcattttgcccaagagttttggccagagcagaacataaaagattcttttgaaaaagtgacactgagaagatatgaaaaatgtggaaatgataattttcagttaaaaggctgtaaaagtgtggatgaatgtaaggtgcacaagggaggttataatggacttaaccaatgtttaccaactatgcagagcaaaatgtttcaatgtgatgaatatgtgaaagtctttaataaattttcacattcagatagacataagataaaacatatggaaaataaacctttcaaatgtaaagaatgtggcagatcgttttgcatgctttcacacctaactcgacatgaaagaaattataccaaggtgaatttctgcaaatgtgaagaatgtgaaaaagctgttaaccaatcttcaaagcttactaaacataaaagaatttatacttgtgagaaactctacaaatgtcaagaatgtgacagaacttttaaccaattctcaaaccttactgaatataaaaaagattatgctcgagagaaaccatacaaatgtgaagaatgtggcaaagcctttaaccagtcctcacaccttactacacataagataattcatacaggagagaaaccctacaaatgtgaagaatgcggcaaagcctctaaccagttctcaaatcttactacacataaaaaaattcatactggagagcaaccctacatatgtgaagaatgtggcaaggcttttacccaatcctcaacccttactacacataagagaattcatactggagaaaaaccctacaaatgtgaagaatgtggaaaagcttttaaccgatcctcaaaacttactgaacataaaaacattcatactggagagcaaccctacaaatgtgaggaatgcggcaaagcttttaaccgatcctcaaatcttacggaacataggaaaattcataccgaagagaaaccctacaaatgtaaagaatgtggcaaagcctttaaacactcctcagcccttactacacataagagaattcacactggagagaaaccctacaaatgtgaagaatgtggcaaagcttttaaccgatcctcaaaacttactgaatataagaaacttcatactggaaagaaaccctacaaatgtgaagaatgtggcaaagcttttatccaatcctcaaaacttactgaacataagaaaattcattctggagagataccctacaagtgtgaagaatgtggcaaagcttttaaacactcctcatcccttactacacataaaagaattcatactggggagaaaccctacaaatgtgaagaatgtagcaaagcttttagccgatcctcaaaacttactgaacataagataattcatactggagagaaaccctatgaatgtgagagatgtgacaaagcttttaaccaatctgcaaaccttactaaacataaaaaaatacatactggagagaaactacagaactggaatgtgtgataatgattttgacaacacctcaaat

SEQ ID NO: 7 - protein, 568 aa, Homo sapiens
MGLLTFRDVAIEFSLEEWQCLDTAQRNLCKNVILENYRNLVFLGIAVSKQDLITCLEQEKEPLTVKRHEMVNEPPVMCSHFAQEFWPEQNIKDSFEKVTLRRYEKCGNDNFQLKGCKSVDECKVHKGGYNGLNQCLPTMQSKMFQCDEYVKVFNKFSHSDRHKIKHMENKPFKCKECGRSFCMLSHLTRHERNYTKVNFCKCEECEKAVNQSSKLTKHKRIYTCEKLYKCQECDRTFNQFSNLTEYKKDYAREKPYKCEECGKAFNQSSHLTTHKIIHTGEKPYKCEECGKASNQFSNLTTHKKIHTGEQPYICEECGKAFTQSSTLTTHKRIHTGEKPYKCEECGKAFNRSSKLTEHKNIHTGEQPYKCEECGKAFNRSSNLTEHRKIHTEEKPYKCKECGKAFKHSSALTTHKRIHTGEKPYKCEECGKAFNRSSKLTEYKKLHTGKKPYKCEECGKAFIQSSKLTEHKKIHSGEIPYKCEECGKAFKHSSSLTTHKRIHTGEKPYKCEECSKAFSRSSKLTEHKIIHTGEKPYECERCDKAFNQSANLTKHKKIHTGEKLQNWNV

SEQ ID NO: 8 - protein, 595 aa, Homo sapiens
MGPLTFRDVAIEFSLKEWQCLDTAQRNLYRNVMLENYRNLVFLGITVSKPDLITCLEQGKEAWSMKRHEIMVAKPTVMCSHFARDLWPEQNIKDSFQKVTLKRYGKCRHENLPLRKGCESMDECKMHKGGCNGLNQCLTATQSKIFQCDKYVKVAHKFSNSNRHEIRHTKKKPFKCTKCGKSFGMISCLTEHSRIHTRVNFYKCEECGKAFNWSSTLTKHKRIHTCEKPYKCEECGKAFNQSSNLIKHKKIHTGEKPYKCEECGKAFNRFSTLTTHKIIHTGEKPYKCKECGKAFNRSSTLTTHRKIHTGEKPYKCEECGKAFKQSSNLTTHKIIHTGEKPYKCKKCGKAFNQSAHLLTHEVIHTGEKPYKCEKCGKAFNHFSHLTTHKIIHTGEKPYKCKECGKAFKHSSTLTKHKIIHTGEKPYKSKECEKAFNQSSKLTEHKKIHTGEKPYECEKCGKAFNQSSNLTRHKKSHTEEKPYKCEECGKGFKWPSTLTIHKIIHTGEKPYKCEECGKAFNQSSKLTKHKKIHTGEKPYTCEECGKAFNQSSNLTKHKRIHTGEKPYKCEECDKAFKWSSVLTKHKIIHTGEKLQI

SEQ ID NO: 9 - protein, 12 aa, Artificial
  Similarity between TBZF and znf 58
LTFRDVAIEFSL

SEQ ID NO: 10 - protein, 12 aa, Artificial
  Similarity between TBZF and znf 58
EWQCLDTAQRNL

SEQ ID NO: 11 - protein, 12 aa, Artificial
  Similarity between TBZF and znf 58
LENYRNLVFLGI

SEQ ID NO: 12 - protein, 8 aa, Artificial
  Similarity between TBZF and znf 58
DLITCLEQ

SEQ ID NO: 13 - protein, 4 aa, Artificial
  Similarity between TBZF and znf 58
KRHE

SEQ ID NO: 14 - protein, 7 aa, Artificial
  Similarity between TBZF and znf 58
VMCSHFA

SEQ ID NO: 15 - protein, 10 aa, Artificial
  Similarity between TBZF and znf 58
WPEQNIKDSF

SEQ ID NO: 16 - protein, 4 aa, Artificial
  Similarity between TBZF and znf 58
KVTL

SEQ ID NO: 17 - protein, 4 aa, Artificial
  Similarity between TBZF and znf 58
DECK

SEQ ID NO: 18 - protein, 4 aa, Artificial
  Similarity between TBZF and znf 58
HKGG

SEQ ID NO: 19 - protein, 7 aa, Artificial
  Similarity between TBZF and znf 58
NGLNQCL

SEQ ID NO: 20 - protein, 4 aa, Artificial
  Similarity between TBZF and znf 58
FQCD

SEQ ID NO: 21 - protein, 4 aa, Artificial
  Similarity between TBZF and znf 58
YVKV

SEQ ID NO: 22 - protein, 5 aa, Artificial
  Similarity between TBZF and znf 58
KPFKC

SEQ ID NO: 23 - protein, 5 aa, Artificial
  Similarity between TBZF and znf 58
KCEEC

SEQ ID NO: 24 - protein, 7 aa, Artificial
  Similarity between TBZF and znf 58
LTKHKRI

SEQ ID NO: 25 - protein, 14 aa, Artificial
  Similarity between TBZF and znf 58
EKPYKCEECGKAFN

SEQ ID NO: 26 - protein, 16 aa, Artificial
  Similarity between TBZF and znf 58
LTTHKIIHTGEKPYKC

SEQ ID NO: 27 - protein, 5 aa, Artificial
  Similarity between TBZF and znf 58
ECGKA

SEQ ID NO: 28 - protein, 4 aa, Artificial
  Similarity between TBZF and znf 58
LTTH

SEQ ID NO: 29 - protein, 6 aa, Artificial
  Similarity between TBZF and znf 58
KIHTGE

SEQ ID NO: 30 - protein, 8 aa, Artificial
  Similarity between TBZF and znf 58
CEECGKAF

SEQ ID NO: 31 - protein, 5 aa, Artificial
  Similarity between TBZF and znf 58
LTTHK

SEQ ID NO: 32 - protein, 10 aa, Artificial
  Similarity between TBZF and znf 58
IHTGEKPYKC

SEQ ID NO: 33 - protein, 6 aa, Artificial
  Similarity between TBZF and znf 58
CGKAFN

SEQ ID NO: 34 - protein, 5 aa, Artificial
  Similarity between TBZF and znf 58
IHTGE

SEQ ID NO: 35 - protein, 5 aa, Artificial
  Similarity between TBZF and znf 58
PYKCE

SEQ ID NO: 36 - protein, 6 aa, Artificial
  Similarity between TBZF and znf 58
CGKAFN

SEQ ID NO: 37 - protein, 17 aa, Artificial
  Similarity between TBZF and znf 58
EKPYKCKECGKAFKHSS

SEQ ID NO: 38 - protein, 9 aa, Artificial
  Similarity between TBZF and znf 58
IHTGEKPYK

SEQ ID NO: 39 - protein, 4 aa, Artificial
  Similarity between TBZF and znf 58
KAFN

SEQ ID NO: 40 - protein, 6 aa, Artificial
  Similarity between TBZF and znf 58
SSKLTE

SEQ ID NO: 41 - protein, 5 aa, Artificial
  Similarity between TBZF and znf 58
CGKAF

SEQ ID NO: 42 - protein, 9 aa, Artificial
  Similarity between TBZF and znf 58
PYKCEECGK

SEQ ID NO: 43 - protein, 13 aa, Artificial
  Similarity between TBZF and znf 58
IHTGEKPYKCEEC

SEQ ID NO: 44 - protein, 5 aa, Artificial
  Similarity between TBZF and znf 58
SSKLT

SEQ ID NO: 45 - protein, 8 aa, Artificial
  Similarity between TBZF and znf 58
IHTGEKPY

SEQ ID NO: 46 - protein, 6 aa, Artificial
  Similarity between TBZF and znf 58
KAFNQS

SEQ ID NO: 47 - protein, 6 aa, Artificial
  Similarity between TBZF and znf 58
NLTKHK

SEQ ID NO: 48 - protein, 6 aa, Artificial
  Similarity between TBZF and znf 58
IHTGEK

SEQ ID NO: 49 - DNA, 24 bp, Artificial
  Primer amplifying TRAF6 inhibiting protein
gttaacagctttttcacattcttc

SEQ ID NO: 50 - DNA, 24 bp, Artificial
  Primer for amplifying TRAF6 inhibiting protein
ggccagagcagaaccataaaagat

SEQ ID NO: 51 - DNA, 27 bp, Artificial
  Primer for amplifying TRAF-6 inhibiting protein
cgggatcccgcataagataaaacatat

SEQ ID NO: 52 - DNA, 26 bp, Artificial
  Primer for amplifying TRAF6 inhibiting protein
aactgcagtcacacattccagttctg